(19) United States
(12) Patent Application Publication
(10) Pub. No.: US 2013/0137154 A1
(43) Pub. Date: May 30, 2013

(54) Systems and Methods for Developing Terrestrial and Algal Biomass Feedstocks and Bio-Refining the Same

(71) Applicant: ORIGINOIL, INC., Los Angeles, CA (US)

(72) Inventor: Paul Reep, Marina Del Ray, CA (US)

(73) Assignee: ORIGINOIL, INC., Los Angeles, CA (US)

(21) Appl. No.: 13/652,250

(22) Filed: Oct. 15, 2012

Related U.S. Application Data
(63) Continuation of application No. 61/547,200, filed on Oct. 14, 2011.

(51) Int. Cl. C10L 1/02; C10L 5/44; C10J 3/00; C10B 57/00; C10G 1/00; C07C 1/20; C12P 3/00; C11B 1/10; C10L 1/18; C10G 1/06

(52) U.S. Cl. CPC C10L 1/02; C10L 1/1802; C10L 5/447; C10J 3/00; C10G 1/06; C10G 1/002; C07C 1/20; C12P 3/00; C11B 1/10; C10B 57/00
USPC 435/168; 44/388; 44/307; 44/605; 48/197 FM; 585/240; 585/310; 585/733; 554/8; 366/348; 366/162.1; 201/21

(57) ABSTRACT

Methods and systems for developing and bio-refining or processing biomass feedstocks into a spectrum of bio-based products which can be used as a substitute for fossil oil derivatives in various types of product manufacturing processes and/or the production of bio-energy are disclosed. In addition, methods and systems for identifying, measuring and controlling key parameters in relation to specific biomass developing processes and bio-refining processes so as to maximize the efficiency and efficacy of such processes while standardizing the underlying parameters to facilitate and enhance large-scale production of bio-based products and/or bio-energy are disclosed.

### RELATED APPLICATIONS

This application claims priority to U.S. Provisional Patent Application Ser. No. 61/547,200 filed Oct. 14, 2011, entitled SYSTEMS AND METHODS FOR DEVELOPING TERRESTRIAL AND ALGAL BIOMASS FEEDSTOCKS AND BIO-REFINING THE SAME.

### FIELD AND BACKGROUND OF THE INVENTION

1. Field of the Invention

The present invention relates to the fields of energy and microbiology. In particular, the invention relates to systems and methods for developing and bio-refining or processing biomass feedstocks into a spectrum of bio-based products which can be used as a substitute for fossil oil derivatives in various types of product manufacturing processes and/or the production of bio-energy. The present invention further relates to systems and methods for identifying, measuring and controlling key parameters in relation to specific biomass developing processes and bio-refining processes so as to maximize the efficiency and efficacy of such processes while standardizing the underlying parameters to facilitate and enhance large-scale production of bio-based products and/or bio-energy.

2. Background and Related Art

Products which may be derived from biomass, such as the intracellular products of microorganisms, show promise as partial or full substitutes for fossil oil derivatives or other chemicals used in manufacturing products such as, inter alia, pharmaceuticals, cosmetics, nutraceuticals, other food products, industrial products, biofuels, synthetic oils, animal feed, fertilizers and so forth. However, for these substitutes to become viable, methods for both fostering the growth and development of the biomass and obtaining and processing usable bio-based products must be efficient and cost effective in order to be competitive with the refining costs associated with fossil oil derivatives. Current systems and methods used for harvesting bio-based products for use as fossil oil substitutes are laborious and yield low net energy gains, rendering them unfeasible for today's alternative energy demands. Further, such methods can produce a significant carbon footprint, exacerbating global warming and other environmental issues. These methods, when further scaled up, produce an even greater efficiency loss, due to valuable intracellular component degradation, and require greater energy or chemical inputs than what is currently financially and/or environmentally feasible from a commercially viable biomass harvest.

Recovery of intracellular particulate substances or products from biomass sometimes requires disruption, lysing or fracturing of the cell transmembrane. Intracellular extraction methods can vary greatly depending on the type of organism involved, their desired internal component(s), and their purity levels. However, once the cell has been fractured, these useful components are released and typically suspended within a liquid medium which is used to house a living microorganism biomass, making harvesting these useful substances difficult and/or energy intensive.

For example, in most current methods of harvesting intracellular products from algae, a dewatering process must be implemented in order to separate and harvest useful components from a liquid medium or from biomass waste (cellular mass and debris). Current processes are inefficient due to the required time lapse for liquid evaporation, energy inputs required for drying out a liquid medium or chemical inputs needed for substance separation.

In addition, as mentioned above, several unique challenges arise in the context of large-scale biomass feedstock development and refining processes. For example, large-scale processes often require that the associated biomass feedstock be transported and/or stored. Current methods for developing biomass feedstocks and subsequently processing or refining the same result in feedstocks and/or bio-based products which are expensive to transport and store or which have an insufficient shelf life to accommodate such transportation and/or storage. Moreover, current methods are not standardized or uniform rendering such methods unsuitable for large-scale expansion of emerging bio-energy industries comprised of networks of biomass growers and refiners and associated infrastructure.

Accordingly, there is a need for a simple, efficient and uniform procedure for the development of biomass feedstocks and the refinement of such feedstocks to develop a spectrum of bio-based products that can be used as competitively-priced substitutes for fossil oils and fossil oil derivatives required for manufacturing processes and energy production.

### BRIEF SUMMARY OF THE INVENTION

The present invention relates to the fields of energy and microbiology. In particular, implementation of the invention relates to systems and methods for developing and bio-refining or processing biomass feedstocks into a spectrum of bio-based products which can be used as a substitute for fossil oil derivatives in various types of product manufacturing processes and/or the production of bio-energy. Various implementations further relate to systems and methods for identifying, measuring and controlling key parameters in relation to specific biomass developing processes and bio-refining processes so as to maximize the efficiency and efficacy of such processes while standardizing the underlying parameters to facilitate and enhance large-scale production of bio-based products and/or bio-energy.

Further implementations relate to systems and methods for the efficient and uniform development of biomass feedstocks and the refinement of such feedstocks to extract useful bio-products therefrom. Various implementations contemplate the development of diverse biomass feedstocks including terrestrial biomass, such as grain and other herbaceous biomass as well as woody and other lignocellulosic biomass, and other high moisture content biomass, such as algae. Further implementations relate to formatting such feedstocks, either during development or following feedstock growth, such that the resulting feedstock is suitably formatted for immediate and/or direct use in various applications, processes or technologies. Alternatively, various feedstocks are formatted to facilitate further downstream refinements in some implementations.

According to certain implementations, one or more feedstocks, which might otherwise be considered pure or homogeneous, can be mixed, blended or combined with other feedstocks to enhance certain characteristics or properties of the resulting mixture. In some implementations, the moisture content of the various feedstocks or feedstock mixtures is also or alternatively subject to adjustment to further enhance various characteristics or properties of such feedstocks or feedstock mixtures.

As mentioned above, according to some implementations, formatted feedstocks are useful for carrying out or facilitating one or more subsequent processes, applications or technologies for producing a spectrum of bio-based products which can be used as substitutes for fossil oils and fossil oil derivatives in product manufacturing processes and/or the production of bio-energy. Some further implementations contemplate the standardization of certain feedstocks and associated parameters, such as blend, moister content, format, and/or other properties and characteristics of such feedstocks, so as to facilitate large-scale processes, applications or technologies in order to optimize and efficiently scale-up the production of a spectrum of useful bio-based products.

According to some implementations, additional systems and methods are contemplated for developing and/or processing certain biomass feedstocks. For example, in some implementations, the present invention relates to extracting intracellular products from microalgae, including lipids, and to the lipid products extracted from these systems and methods. In such implementations, the systems and methods of the invention can advantageously extract valuable intracellular products from microalgae at a high volume flow rate. By separating non-polar lipids (e.g., triglycerides) from polar lipids (e.g., phospholipids and chlorophyll) and cellular debris, the methods and systems of the invention can produce a product suitable for use in traditional petrochemical processes, such as petrochemical processes that utilize precious metal catalysts.

In various implementations, the present invention relates to a method for extracting intracellular products from microalgae in a flowing aqueous slurry. The method includes (i) providing an aqueous slurry including microalgae; (ii) providing a lipid extraction apparatus having a body including a channel that defines a fluid flow path, at least a portion of the channel formed from a cathode and an anode spaced apart to form a gap; (iii) flowing the aqueous slurry through the channel and applying an electromotive force across the gap, the electromotive force compromising the microalgae cells and releasing various intracellular products therefrom; and (iv) recovering at least a portion of the intracellular products.

According to some further implementations, the present invention relates to systems and methods for identifying and measuring key parameters of water and gas chemistry in which algae cells can grow and be mass produced such that when the algae cells mature they are separable from the water and the algae cells can be fractured in order to separate cellular mass and debris from various intracellular products using pulsed electromotive forces (hereinafter “emf”) or electromagnetic pulses (hereinafter “EMP”) and other methods, including mechanical and/or chemical. In addition, it is contemplated that other process chemistry parameters, such as various product/byproduct gasses generated largely by process reaction, may be identified and measured. Such measurement and control parameters are useful in the complete life cycle of algae growth, algae cell death, water/oil/biomass separation, reticulating/reusing process water and/or nutrients, and prescribing additional nutrients, additives and/or admixtures for the feed water system, such as carbon dioxide (CO₂).

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates a portion of a lipid extraction device according to one embodiment of the invention;

FIG. 2 illustrates a sectional perspective view of biomass flowing in between the anode and cathode wall surfaces of the device of FIG. 1;

FIG. 3 illustrates a lipid extraction apparatus with a flowing liquid medium containing a microorganism biomass being exposed to an electromagnetic field caused by an electrical transfer;

FIG. 4 illustrates an overview of a normal sized microorganism cell in relationship to a secondary illustration of a swollen cell during exposure to an electromagnetic field and electrical charge;

FIG. 5 illustrates the lipid extraction apparatus of FIG. 3 with heat being applied and transferred into the liquid medium;

FIG. 6 illustrates a perspective view of the anode and cathode tubes of an apparatus according to one embodiment of the invention;

FIG. 7 illustrates a perspective sectional view of the apparatus of FIG. 6 including a spiral spacer in between the anode and cathode tubes;

FIG. 8 is a perspective view of a series of lipid extraction devices of FIG. 7 connected in parallel by an upper and lower manifold;

FIG. 9 depicts a general flow diagram illustrating various steps of a process for extracting intracellular products from microalgae according to one embodiment of the present invention;

FIG. 10 depicts a general flow diagram illustrating various steps of a process for extracting intracellular products from microalgae according to another embodiment of the present invention;

FIG. 11 illustrates a side view of a micron mixer in association with a secondary tank containing a biomass and sequences of developing foam layers generated by a micron mixer;

FIG. 12 illustrates a secondary tank containing the liquid medium and a resulting foam layer capable of being skimmed off the surface of the liquid medium, into a foam harvest tank;

FIG. 13 illustrates one embodiment of a method and apparatus (system) as described herein for the harvest of useful substances from an algae biomass involving single step extraction;

FIG. 14 illustrates another embodiment of a method and apparatus (system) as described herein for the harvest of useful substances from an algae biomass using a lipid extraction device that applies emf;

FIG. 15 illustrates an example of a modified static mixer;

FIG. 16 illustrates a non-limiting example of various sensor components according to some embodiments of the present invention;

FIG. 17 illustrates a non-limiting example of Supervisory Control and Data Acquisition components according to certain embodiments of the present invention;

FIG. 18 illustrates a non-limiting example of a system according to some embodiments of the present invention; and

FIGS. 19A and 19B illustrate a front and top view respectively of a non-limiting example of a sensor array according to various embodiments of the present invention.

### DETAILED DESCRIPTION OF THE INVENTION

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The following disclosure of the present invention may be grouped into subheadings. The utilization of the subheadings is for convenience of the reader only and is not to be construed as limiting in any sense.

The description may use perspective-based descriptions such as up/down, back/front, left/right and top/bottom. Such descriptions are merely used to facilitate the discussion and are not intended to restrict the application or embodiments of the present invention.

For the purposes of the present invention, the phrase “A/B” means A or B. For the purposes of the present invention, the phrase “A and/or B” means “(A), (B), or (A and B).” For the purposes of the present invention, the phrase “at least one of A, B, and C” means “(A), (B), (C), (A and B), (A and C), (B and C), or (A, B and C).” For the purposes of the present invention, the phrase “(A)B” means “(B) or (AB)”, that is, A is an optional element.

Various operations may be described as multiple discrete operations in turn, in a manner that may be helpful in understanding embodiments of the present invention; however, the order of description should not be construed to imply that these operations are order dependent.

The description may use the phrases “in an embodiment,” or “in various embodiments,” which may each refer to one or more of the same or different embodiments. Furthermore, the terms “comprising,” “including,” “having,” and the like, as used with respect to embodiments of the present invention, are synonymous with the definition afforded the term “comprising.”

The terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical or electrical contact. However, “coupled” may also mean that two or more elements are not in direct contact with each other, but yet still cooperate or interact with each other.

The present invention relates to the fields of energy and microbiology. In particular, embodiments of the invention relate to systems and methods for developing and bio-refining or processing biomass feedstocks into a spectrum of bio-based products which can be used as a substitute for fossil oil derivatives in various types of product manufacturing processes and/or the production of bio-energy. Various embodiments further relate to systems and methods for identifying, measuring and controlling key parameters in relation to specific biomass developing processes and bio-refining processes so as to maximize the efficiency and efficacy of such processes while standardizing the underlying parameters to facilitate and enhance large-scale production of bio-based products and/or bio-energy.

Further embodiments relate to systems and methods for the efficient and uniform development of biomass feedstocks and the refinement of such feedstocks to extract useful bio-products therefrom. Various embodiments contemplate the development of diverse biomass feedstocks including terrestrial biomass, such as grain and other herbaceous biomass as well as woody and other lignocellulosic biomass, and other high moisture content biomass, such as algae. Further embodiments relate to formatting such feedstocks, either during development or following feedstock growth, such that the resulting feedstock is suitably formatted for immediate and/or direct use in various applications, processes or technologies. Alternatively, various feedstocks are formatted to facilitate further downstream refinements in some embodiments.

According to certain embodiments, one or more feedstocks, which might otherwise be considered pure or homogeneous, can be mixed, blended or combined with other feedstocks to enhance certain characteristics or properties of the resulting mixture. In some embodiments, the moisture content of the various feedstocks or feedstock mixtures is also or alternatively subject to adjustment to further enhance various characteristics or properties of such feedstocks or feedstock mixtures.

As mentioned above, according to some embodiments, formatted feedstocks are useful for carrying out or facilitating one or more subsequent processes, applications or technologies for producing a spectrum of bio-based products which can be used as substitutes for fossil oils and fossil oil derivatives in product manufacturing processes and/or the production of bio-energy. Some further embodiments contemplate the standardization of certain feedstocks and associated parameters, such as blend, moister content, format, and/or other properties and characteristics of such feedstocks, so as to facilitate large-scale processes, applications or technologies in order to optimize and efficiently scale-up the production of a spectrum of useful bio-based products.

According to some embodiments, additional systems and methods are contemplated for developing and/or processing certain biomass feedstocks, including lipid feedstocks. For example, in some embodiments, the present invention relates to extracting intracellular products from microalgae, including lipids, and to the lipid products extracted from these systems and methods. In such embodiments, the systems and methods of the invention can advantageously extract valuable intracellular products from microalgae at a high volume flow rate. By separating non-polar lipids (e.g., triglycerides) from polar lipids (e.g., phospholipids and chlorophyll) and cellular debris, the methods and systems of the invention can produce a product suitable for use in traditional petrochemical processes, such as petrochemical processes that utilize precious metal catalysts.

In various embodiments, the present invention relates to a method for extracting intracellular products from microalgae in a flowing aqueous slurry. The method includes (i) providing an aqueous slurry including microalgae; (ii) providing a lipid extraction apparatus having a body including a channel that defines a fluid flow path, at least a portion of the channel formed from a cathode and an anode spaced apart to form a gap; (iii) flowing the aqueous slurry through the channel and applying an electromotive force across the gap, the electromotive force compromising the microalgae cells and releasing various intracellular products therefrom; and (iv) recovering at least a portion of the intracellular products.

According to some embodiments, the present invention further relates to systems and methods for identifying and measuring key parameters of water and gas chemistry in which algae cells can grow and be mass produced such that when the algae cells mature they are separable from the water and the algae cells can be fractured in order to separate cellular mass and debris from various intracellular products using pulsed electromotive forces (hereinafter “emf”) or electromagnetic pulses (hereinafter “EMP”) and other methods, including mechanical and/or chemical. In addition, it is contemplated that other process chemistry parameters, such as various product/byproduct gasses generated largely by process reaction, may be identified and measured. Such measurement and control parameters are useful in the complete life cycle of algae growth, algae cell death, water/oil/biomass separation, reticulating/reusing process water and/or nutrients, and prescribing additional nutrients, additives and/or admixtures for the feed water system, such as carbon dioxide (CO₂).

### Cultivating Biomass Feedstocks

As mentioned briefly above, some embodiments contemplate the development of diverse biomass feedstocks. According to some embodiments, the development of the underlying biomass includes fostering, nurturing, cultivating, collecting, maturing or otherwise growing the biomass. In other embodiments, discussed in further detail below, the development of biomass feedstocks includes blending or mixing underlying or otherwise discrete biomass feedstocks having similar or dissimilar properties, characteristics, chemical formulations, chemical structures and/or chemical functional groups. In still other embodiments, also discussed in greater detail below, the development of biomass feedstocks includes formatting such feedstocks and/or feedstock mixtures such that the same are suitable for subsequent processing and/or use. In some embodiments, uniform standards are contemplated for both blending as well as formatting various feedstocks and/or feedstock mixtures such that the resulting feedstocks and/or feedstock compositions are biochemically and physically uniform industry wide. In some additional embodiments, it is contemplated that the resulting feedstocks and/or feedstock compositions are also uniform from a chemical functional grouping standpoint. In this way, the uniform feedstocks and/or feedstock compositions are suitable for large-scale refinement and/or subsequent processing so as to maximize the efficiency and yield of such refinement and/or processes.

In some embodiments, the biomass is comprised of terrestrial biomass, such as grain and other herbaceous biomass as well as woody and other lignocellulosic biomass (i.e., plant biomass, plant-derived organic matter or polymeric structures primarily comprised of, inter alia, cellulose, hemicellulose and lignin). In general, according to various embodiments, biomass available for use as feedstocks include herbaceous and woody energy crops, agricultural food and feed crops, agricultural crop wastes and residues, wood wastes and residues, aquatic plants and other waste materials including some municipal wastes.

For example, in various embodiments, terrestrial biomass includes, but is not limited to, woods, wood residues and/or fast growing wood plants and other woody materials, such as poplar, willow, elephant grass, eucalyptus, bamboo, beech, spruce, pine, black locust robinia, sycamore and/or various species of each of the foregoing, as well as sawmill and/or paper mill discards, such as sawdust, bark, shavings, spent pulping liquors, cellulose sludge or other wood wastes and/or wood residues. In other embodiments, terrestrial biomass includes, but is not limited to herbaceous materials, such as dedicated energy corps as well as agricultural food and/or feed crops, such as fruits, sugar cane, corn, sugar beets, palm juice, switchgrass, tall fescue, sweet sorghum, forage sorghum, big bluestem, little bluestem, sericea lespedeza, indiangrass, prairie cordgrass, miscanthus, intermediate wheatgrass, reed canarygrass, smooth bromegrass, hay, timothyb, alfalfa, pearl millet, sudangrass and other dedicated perennial grasses, wheat and other small grains, soy beans, potatoes, cassaya and the like. In still other embodiments, terrestrial biomass includes agricultural residues and/or crop wastes, such as corn stover, soybean residues, sugarcane bagasse, corn stalk, corn leaf, rice husk, rice straw, wheat straw, rye straw, barley straw and other agricultural wastes or residues. In some additional embodiments, terrestrial feedstocks include vegetable and/or other oils such as palm, sunflower, and rapeseed, organic wastes such as livestock manures including swine manure and dairy manure, municipal waste such as paper waste, solid waste, garbage or refuse, sewage sludge and other industrial wastes such as plastics and aluminum. Additional terrestrial biomass feedstock materials are contemplated herein, the foregoing list being merely illustrative and not limiting.

In other embodiments, the biomass and/or biomass feedstocks are comprised of high moisture content biomass, such as algae. In some embodiments, an algae slurry comprising ten percent (10%) water is contemplated. In other embodiments, an algae slurry comprising twenty percent (20%) water is contemplated. In yet other embodiments, an algae slurry comprising thirty percent (30%) water is contemplated. In some embodiments, an algae slurry comprising forty percent (40%) water is contemplated. In some embodiments, the moister content of the algae slurry ranges from one present (1%) to forty percent (40%). In still other embodiments, algae having a low moisture content comprise a useful biomass feedstock. In various additional embodiments, other high moisture content biomass feedstocks include beet pulp, sludge, and similar high moisture content biomass.

In embodiments contemplating a biomass feedstock comprised of or including algae, various algae and/or microalgae are contemplated including algae suitable for energy stock, agricultural feed, and/or food grade algae, autotrophic algae, heterotrophic algae and so forth. For example, in various embodiments, the algae cells can be any cells, including, but not limited to, Nanochloropsis oculata, Scenedesmus, Chlamydomonas, Chlorella, Spirogyra, Euglena, Prymnesium, Porphyridium, Synechoccus, Cyanobacteria and certain classes of Rhodophyta single celled strains.

In various embodiments contemplating the cultivation of any or all of the foregoing biomass feedstocks, various methods, common to those of skill in the art, may be employed to foster, nurture, cultivate, collect, mature or otherwise grow the biomass feedstocks. For example, various methods can be employed to enhance and foster the growth of algae in water while other methods can be employed to enhance or otherwise facilitate the growth of switchgrass and so forth. In still other embodiments, a feedstock, such as municipal waste or sludge, can be collected from various sources.

### Blending Biomass Feedstocks

In various embodiments, biomass feedstocks are comprised of a single homogeneous biomass. In such embodiments, the feedstock may be considered a pure feedstock comprised of a single biomass. In other words, in some embodiments, certain feedstocks are well suited for subsequent processing or refinement in their pure form. For example, in some embodiments, algae are used as a feedstock independent of any other feedstocks. In other words, in such embodiments, algal biomass feedstock is used alone. In some embodiments, pure or otherwise homogeneous biomass feedstocks are combined with water to facilitate development of the feedstock and/or the subsequent processing of the feedstock. In various embodiments, uniform standards are contemplated for the development of homogeneous feedstocks. For example, in embodiments contemplating the use of an algal slurry, the slurry can be cultivated so as to have a specified water content or lipid content. In other embodiments, the presence of chemicals, such as chemical coagulants or flocculants, can be standardized. In some embodiments, it is contemplated that no chemicals are included in the slurry or the slurry is chemical free throughout development, harvesting and processing of the same.

In other embodiments, however, one or more feedstocks, which might otherwise be considered pure or homogeneous, respectively, are mixed, blended or combined with other feedstocks to enhance certain characteristics or properties of the resulting mixture or feedstock composition. In other words, in some embodiments, certain feedstocks are not well suited for subsequent processing or refinement in their pure form but through blending such feedstocks with other feedstocks the resulting mixture can be optimized for specific downstream processes. In some embodiments, the moisture content of the feedstock compositions is subject to adjustment to further enhance various characteristics or properties of such feedstock mixtures. In still other embodiments, feedstock compositions are created by combining one or more base feedstocks with various additives or add mixtures. In some embodiments, such additives include various strains of bacteria and/or catalysts configured to facilitate the growth and/or processing or refining of the feedstock and/or feedstock compositions. In other embodiments, such additives comprise ethanol, sodium, and/or ethanolate. In some embodiments, for example, algae feedstocks are combined with herbaceous feedstocks. In other embodiments, for example, algae feedstocks are combined with woody feedstocks. In some embodiments, algae feedstocks act as either a binder or a catalyst such that mixing algae with either herbaceous or woody feedstocks renders the later suitable, or otherwise improves the properties of the latter, with respect to certain downstream processes.

In the various embodiments, the feedstock compositions can be manipulated to adjust the ratio of one underlying feedstock biomass to another underlying feedstock biomass and/or to adjust the ratio of additives to feedstocks and/or to adjust the ratio of feedstocks to water or moisture content. In this way, as mentioned above, various characteristics or properties of the feedstock compositions can be enhanced or otherwise adjusted. For example, properties or characteristics such as energy content or value, transportability, longevity, shelf-life, solubility, density and the like are adjustable according to the feedstock composition selected by a user. In other embodiments, feedstock compositions, blends or mixtures are adjusted to control cost and other considerations.

By way of example and not limitation, in some embodiments, it is desirable to improve the transportability of a feedstock composition such that it may be transported easily and inexpensively. In such embodiments, for example, the moisture content of a given feedstock or feedstock composition, such as a feedstock composition comprising at least some algae biomass, can be increased in order to transport the feedstock composition as a liquid slurry via pipeline. Alternatively, in the foregoing example, the algae slurry could be transported prior to dewatering. In another example, according to some embodiments, it is desirable to improve the shelf-life of a feedstock composition such that it may be stored for a period of time prior to use. In some embodiments, it is preferable to store a feedstock composition for at least one (1) year or longer. In such embodiments, for example, the water or moisture content of a given feedstock composition, such as a feedstock composition comprising at least some algae biomass, can be dewatered or otherwise have the moisture content reduced in order to improve the shelf-life of the feedstock composition. Similar adjustments to the feedstock composition are directed at increasing the density of the composition such that an identical quantity of feedstock can be shipped using less space or consuming less chargeable shipping volume.

### Illustrative Examples

As discussed in general above, systems and methods are contemplated for standardizing homogeneous feedstocks and/or for standardizing feedstock blends or compositions. To further illustrate various principles of such embodiments, the following examples are provided with the understanding that such examples are not intended to be limiting.

As mentioned above, systems and methods are disclosed for standardizing intermediate feedstocks and/or feedstock compositions. In some embodiments, such feedstocks or feedstock compositions are uniform biochemically and physically while in other embodiments such feedstocks or compositions are uniform from a chemical grouping standpoint. In other words, in embodiments contemplating feedstock blends, the presence of specific chemical functional groups present in the constituent elements of any such blend and the respective proportion of such chemical functional groups pertain to creating uniform feedstocks. In some embodiments, the result of standardizing feedstocks and/or feedstock blends is to raise the net energy potential of the feedstock or feedstock blend so as to optimize the feedstock or blend for subsequent refinement.

For example, in some embodiments, it is desirable to generate a uniform standard for developing feedstocks such that the resulting feedstocks are optimized or well suited for various downstream processes designed to convert the feedstocks into usable products, such as various bio-products or bio-energy. Standardizing such feedstocks or blends renders the subsequent processing or refinement of the same more efficient and economical on a large-scale across numerous participants, a common infrastructure and numerous end-users. Likewise, the standardization of feedstocks or feedstock blends serves to optimize or maximize the value of the feedstock—meaning that the feedstock houses or contains the maximum energy potential and/or usable bio-products or is particularly adapted for processes configured to harvest such bio-products or bio-energy. For example, while a given feedstock in its pure form can be refined to produce a certain amount of a refined product, blending the feedstock with other feedstocks can maximize the amount of the refined product with the minimum feedstock. In other words, the percentage yield from a given quantity of feedstock can be increased by blending feedstocks.

With the foregoing in mind, the following examples are provided with the understanding that the structural and chemical composition of various feedstocks is highly variable because of genetic and environmental influences and interactions. In this respect, the following examples are provided for purposes of illustrating the principles previously discussed and are not intended to be limiting in any respect.

As mentioned above, in some embodiments, a given feedstock may be used without the necessity of blending the biomass with other biomass feedstocks. For example, in some embodiments, algal feedstock may be used without blending the algae with other feedstocks. In such embodiments, the algae biomass constitutes one-hundred percent (100%), or nearly one-hundred percent pursuant to permitted tolerances, of the drymass of the feedstock. In some embodiments, algae has the following chemical composition:

| Molecule | Weight | Percent |
|---|---|---|
| C106 | 1272 | 35.8% |
| H263 | 263 | 7.4% |
| O110 | 1760 | 49.6% |
| N16 | 224 | 6.3% |
| P | 32 | 1% |
| Total | 3551 | 100% |

In such embodiments, the chemical functional groups present in the algae are defined by the chemical structure of the algae employed. Specifically, the use of various algae cells are contemplated according to various embodiments, such as Nanochloropsis oculata, Scenedesmus, Chlamydomonas, Chlorella, Spirogyra, Euglena, Prymnesium, Porphyridium, Synechoccus, Cyanobacteria and certain classes of Rhodophyta single celled strains as mentioned above.

In addition to the functional chemical groups present in the algae, in various embodiments, the algae further comprises ten percent (10%) water, twenty percent (20%) water, thirty percent (30%) water and/or forty percent (40%) water. In some embodiments, the moister content of the algae ranges from one percent (1%) to forty percent (40%) as discussed above. In other embodiments, the lipid content of the algae used varies according to the algae cells as understood by those of skill in the art. In still other embodiments, the algae, whether in a slurry or dewatered, includes additional chemical additives or residues, such as chemical flocculants. In other embodiments, the algae biomass is chemical free.

In some further embodiments, wherein the use of algal feedstock is contemplated, the algae cells may be lysed, partially lysed or fractured or used without being lysed. Specifically, in some embodiments, algae cells are lysed prior to subsequent processing, refining or blending of the algae with other feedstocks such that the internal cellular (or intracellular) components, such as internal lipid contents, of the algae are released before hand. In some embodiments, the internal cellular components are released into a liquid or aqueous slurry when the algae cells are lysed or otherwise ruptured. In other embodiments, algae cells are fractured or otherwise partially lysed prior to subsequent refinement or blending such that only a portion of the intracellular components of the algae cells are released before hand. In still other embodiments, the algae cells are used following growth or cultivation without being lysed, either in whole or in part. In other words, according to some embodiments, the algae cells are used with their cellular structure intact and with all of their internal cellular components contained therein. Various methods and systems are discussed in greater detail below for lysing or partially lysing algae cells according to some embodiments.

In additional embodiments, other feedstocks, such as herbaceous or woody feedstocks, are used without the necessity of blending the same with other biomass feedstocks as discussed with reference to algae above. In other embodiments, however, various feedstocks are blended to form feedstock compositions. For example, in some embodiments, algae feedstock is combined with one (1) additional feedstock, such as corn stover, wheat straw, switchgrass or other woody or herbaceous feedstocks as discussed above. In other embodiments, three (3) feedstocks (comprised of either algae, woody or herbaceous feedstocks) are combined. In other embodiments, four (4) feedstocks are combined. In still other embodiments, N feedstocks are combined where N represents any number or an infinite number of feedstock constituents. In the foregoing embodiments, the constituent feedstocks may be combined in equal physical and biochemical proportions while in other embodiments the proportion of each constituent feedstock may vary.

By way of example and not limitation, in some embodiments, algae feedstock is combined with corn stover. In some embodiments, algae and corn stover are combined in equal physical and/or biochemical proportions. In other embodiments, algae and corn stover are combined in unequal proportions, either physically, biochemically or both.

According to some embodiments, in terms of the feedstock drymass or dry matter, untreated corn stover is composed of thirty to fifty percent (30%-50%) cellulose, twenty to forty percent (20%-40%) hemicellulose, ten to twenty-five percent (10%-25%) lignin, three to ten percent (3%-10%) ash and five to thirty percent (5%-30%) other compounds, such as resins, fats, fatty acids, phenolics, phytosterols, salts, minerals and other compounds. In other embodiments, other compounds include various extractives, protein, uronic acids and so forth.

According to some embodiments, cellulose is a carbohydrate that is the principal constituent of terrestrial biomass and forms the structural framework of the biomass. In such embodiments, cellulose it a polymer of glucose with a repeating unit of C₆H₁₀O₅ strung together by β-glycosidic linkages. The β-linkages in cellulose form linear chains that are highly stable and resistant to chemical attack because of the high degree of hydrogen bonding that can occur between chains of cellulose. Hydrogen bonding between cellulose chains makes the polymers more rigid, inhibiting the flexing of the molecules that must occur in the hydrolytic breaking of the glycosidic linkages. Hydrolysis can reduce cellulose to a cellobiose repeating unit, C₁₂H₂₂O₁₁, and ultimately to glucose, C₆H₁₂O₆.

According to various embodiments, Hemicellulose consists of short, highly branched chains of sugars. In contrast to cellulose, which is a polymer of only glucose, in some embodiments, a hemicellulose is a polymer of five different sugars. It contains five-carbon sugars (usually xylose and arabinose) and six-carbon sugars (galactose, glucose, and mannose) and uronic acid. In some embodiments, the sugars are highly substituted with acetic acid. The branched nature of hemicellulose renders it amorphous and relatively easy to hydrolyze to its constituent sugars compared to cellulose. According to some embodiments, when hydrolyzed, the hemicellulose from hardwoods releases products high in xylose (a five-carbon sugar). In other embodiments, the hemicellulose contained in softwoods, yields more six-carbon sugars.

As mentioned above, in some embodiments hemicellulose is a polymer of five different sugars. In such embodiments, xylose is a five-carbon sugar C₅H₁₀0₅. According to some embodiments, xylose is a product of hydrolysis of xylan (a polymer of xylose with a repeating unit of C₅H₈0₄ found in the hemicellulose fraction of biomass). In further embodiments, arabinose is also a five-carbon sugar C₅H₁₀0₅. According to some embodiments, arabinose is a product of hydrolysis of arabinan (a polymer of arabinose with a repeating unit of C₅H₈0₄ found in the hemicellulose fraction of biomass). In still further embodiments, galactose is a six-carbon sugar C₅H₁₂0₆. According to some embodiments, galactose is a product of hydrolysis of galactan (a polymer of galactose with a repeating unit of C₆H₁₀0₅ found in the hemicellulose fraction of biomass). In yet further embodiments, glucose is a simple six-carbon sugar C₆H₁₂0₆. According to some embodiments, glucose is a product of hydrolysis of glucan (a polymer of glucose with a repeating unit of C₆H₁₀0₅ found in the hemicellulose fraction of biomass). In still further embodiments, mannose is also a six-carbon sugar C₆H₁₂0₆. According to some embodiments, mannose is a product of hydrolysis of mannan (a polymer of mannose with a repeating unit of C₆H₁₀0₅ found in the hemicellulose fraction of biomass).

In some embodiments, lignin comprises the major noncarbohydrate, polypenolic structural constituent of terrestrial biomass and other native plant material that encrusts the cell walls and cements the cells together. It is a highly polymeric substance, with a complex, cross-linked, highly aromatic structure of molecular weight about 10,000 derived principally from coniferyl alcohol (C₁₀H₁₂O₃) by extensive condensation polymerization. In some embodiments, lignin has the following chemical formulas: C₉H₁₀O₂, C₁₀H₁₂O₃, C₁₁H₁₄O₄.

In various embodiments, ash constitutes the residue remaining after ignition of a sample determined by a definite prescribed procedure. In some embodiments, for example, ash is comprised of silicon, aluminum, calcium, magnesium, potassium, sodium and or other minerals. Some embodiments of terrestrial biomass also include one to ten percent (1%-10%) acetyl (C₂H₃O). In other embodiments, uronic acid is a simple sugar whose terminal—CH₂OH group has been oxidized to an acid, COOH group. The uronic acids occur as branching groups bonded to hemicelluloses such as xylan.

In some embodiments, corn stover is composed (as a percentage of drymass or dry matter) of thirty to forty-five percent (30%-45%) cellulose or structural glucan, twenty to forty percent (20%-40%) hemicelluloses (or fifteen to thirty percent (15%-30%) xylan, one to ten percent (1%-10%) arabinan, a tenth to five percent (0.1%-5%) galactan, and a tenth to five percent (0.1%-5%) mannan), ten to twenty-five percent (10%-25%) total lignin (or one to four percent (1%-4%) acid soluble lignin and ten to twenty percent (10%-20%) acid insoluble lignin), three to ten percent (3%-10%) ash and five to thirty percent (5%-30%) other compounds, such as crude protein, acid detergent lignin, extractives, uronic acids, acetyl and so forth.

In various embodiments, for example, wherein it is contemplated that two feedstocks are blended or mixed together, such as algae and corn stover, algae and corn stover may be mixed in any suitable proportions. For example, form a biomass drymass equivalency perspective, algae and corn stover, according to some embodiments, are mixed at a ratio of about 1:1, about 1:10, about 1:100, about 1:1000, about 10:1, about 10:100, about 10:1000, about 100:1, about 100:10, about 100:1000, about 1000:1, about 1000:10, and about 1000:100. In alternative embodiments, for example, algae and corn stover are mixed in any ratio between 1:1 and 1:1000 or visa-versa (i.e., between 1000:1 and 1:1). In alternative embodiments, in terms of the drymass or dry matter percentage of the total combined feedstock, algae constitutes from one to ten percent (1%-10%) of the total dry matter while corn stover comprises the remaining percentage of the total dry matter. In other embodiments, algae can constitute from one one-hundreth to ten percent (0.01%-10%), ten to twenty percent (10%-20%), twenty to thirty percent (20%-30%), thirty to forty percent (30%-40%), forty to fifty percent (40%-50%), fifty to sixty percent (50%-60%), sixty to seventy percent (60%-70%), seventy to eighty percent (70%-80%), eighty to ninety percent (80%-90%), and/or ninety to ninety-nine and ninety-nine one-hundredth percent (90%-99.99%) of the total dry matter while corn stover comprises the remaining percentage of the total dry matter.

In other embodiments, for example, algae is combined with one or more other terrestrial biomass feedstocks, such as wheat straw, switchgrass, or poplar for example. In further embodiments, one or more terrestrial biomass feedstocks can be combined with one or more additional terrestrial biomass feedstocks instead of or in addition to being combined with algae. Such combinations can be blended in any suitable proportions.

By way of example, in some embodiments, algal is blended with switchgrass. In some embodiments, switchgrass is composed (as a percentage of drymass or dry matter) of thirty to fifty percent (30%-50%) cellulose or structural glucan, twenty to forty percent (20%-40%) hemicelluloses (or fifteen to thirty percent (15%-30%) xylan, one to ten percent (1%-10%) arabinan, a tenth to five percent (0.1%-5%) galactan, and a tenth to five percent (0.1%-5%) mannan), ten to twenty-five percent (10%-25%) total lignin (or one to five percent (1%-5%) acid soluble lignin and ten to twenty-five percent (10%-25%) acid insoluble lignin), three to fifteen percent (3%-15%) ash and five to thirty percent (5%-30%) other compounds, such as crude protein, acid detergent lignin, extractives, uronic acids, acetyl and so forth.

In various embodiments, for example, wherein it is contemplated that one or more feedstocks are blended or mixed together, such as algae and switchgrass, algae and switchgrass may be mixed in any suitable proportions. For example, form a biomass drymass equivalency perspective, algae and switchgrass, according to some embodiments, are mixed at a ratio of about 1:1, about 1:10, about 1:100, about 1:1000, about 10:1, about 10:100, about 10:1000, about 100:1, about 100:10, about 100:1000, about 1000:1, about 1000:10, and about 1000:100. In alternative embodiments, for example, algae and switchgrass are mixed in any ratio between 1:1 and 1:1000 or visa-versa (i.e., between 1000:1 and 1:1). In alternative embodiments, in terms of the drymass or dry matter percentage of the total combined feedstock, algae constitutes from one to ten percent (1%-10%) of the total dry matter while switchgrass comprises the remaining percentage of the total dry matter. In other embodiments, algae can constitute from one one-hundreth to ten percent (0.01%-10%), ten to twenty percent (10%-20%), twenty to thirty percent (20%-30%), thirty to forty percent (30%-40%), forty to fifty percent (40%-50%), fifty to sixty percent (50%-60%), sixty to seventy percent (60%-70%), seventy to eighty percent (70%-80%), eighty to ninety percent (80%-90%), and/or ninety to ninety-nine and ninety-nine one-hundredth percent (90%-99.99%) of the total dry matter while switchgrass comprises the remaining percentage of the total dry matter.

In further embodiments, three (3) or more feedstocks are blended. For example, in some embodiments, algae, corn stover and switchgrass are blended together. For example, form a biomass drymass equivalency perspective, algae, corn stover and switchgrass, according to some embodiments, are mixed or blended at a ratio of about 1:1:1, about 1:1:10, about 1:1:100, about 1:1:1000, about 1:10:1, about 1:10:10, about 1:10:100, about 1:10:1000, about 1:100:1, about 1:100:10, about 1:100:100, about 1:100:1000, about 1:1000:1, about 1:1000:10, about 1:1000:100, about 1:1000:1000, about 10:1:1, about 10:1:10, about 10:1:100, about 10:1:1000, about 10:10:1, about 10:10:100, about 10:10:1000, about 10:100:1, about 10:100:10, about 10:100:100, about 10:100:1000, about 10:1000:1, about 10:1000:10, about 10:1000:100, about 10:1000:1000, about 100:1:1, about 100:1:10, about 100:1:100, about 100:1:1000, about 100:10:1, about 100:10:10, about 100:10:100, about 100:10:1000, about 100:100:1, about 100:100:10, about 100:100:1000, about 100:1000:1, about 100:1000:10, about 100:1000:100, about 100:1000:1000, about 1000:1:1, about 1000:1:10, about 1000:1:100, about 1000:1:1000, about 1000:10:1, about 1000:10:10, about 1000:10:100, about 1000:10:1000, about 1000:100:1, about 1000:100:10, about 1000:100:100, about 1000:100:1000, 1000:1000:1, 1000:1000:10, and 1000:1000:100. In alternative embodiments, for example, algae, corn stover and switchgrass are mixed in any ratio between 1:1:1 and 1:1:1000, 1:1:1 and 1000:1:1, and 1:1:1 and 1:1000:1 or visa-versa. In alternative embodiments, in terms of the drymass or dry matter percentage of the total combined feedstock, algae constitutes from one to ten percent (1%-10%) of the total dry matter while corn stover and/or switchgrass comprise the remaining percentage of the total dry matter. In other embodiments, algae can constitute from one one-hundreth to ten percent (0.01%-10%), ten to twenty percent (10%-20%), twenty to thirty percent (20%-30%), thirty to forty percent (30%-40%), forty to fifty percent (40%-50%), fifty to sixty percent (50%-60%), sixty to seventy percent (60%-70%), seventy to eighty percent (70%-80%), eighty to ninety percent (80%-90%), and/or ninety to ninety-nine and ninety-nine one-hundredth percent (90%-99.99%) of the total dry matter while corn stover and/or switchgrass comprises the remaining percentage of the total dry matter.

Similar examples are contemplated for combinations with wheat straw, wherein wheat straw is composed (as a percentage of drymass or dry matter) of thirty to fifty percent (30%-50%) cellulose or structural glucan, twenty to forty percent (20%-40%) hemicelluloses (or fifteen to thirty percent (15%-30%) xylan, one to ten percent (1%-10%) arabinan, a tenth to five percent (0.1%-5%) galactan, and a tenth to five percent (0.1%-5%) mannan), ten to twenty-five percent (10%-25%) total lignin (or one to five percent (1%-5%) acid soluble lignin and ten to twenty-five percent (10%-25%) acid insoluble lignin), three to fifteen percent (3%-15%) ash and five to thirty percent (5%-30%) other compounds, such as crude protein, acid detergent lignin, extractives, uronic acids, acetyl and so forth.

Another example is contemplated for combinations with poplar, wherein polar is composed (as a percentage of drymass or dry matter) of thirty to fifty percent (30%-50%) cellulose or structural glucan, twenty to forty percent (20%-40%) hemicelluloses (or fifteen to thirty percent (15%-30%) xylan, one to ten percent (1%-10%) arabinan, a tenth to five percent (0.1%-5%) galactan, and a tenth to five percent (0.1%-5%) mannan), ten to thirty percent (10%-30%) total lignin (or one to five percent (1%-5%) acid soluble lignin and ten to thirty percent (10%-30%) acid insoluble lignin), three to fifteen percent (3%-15%) ash and five to thirty percent (5%-30%) other compounds, such as crude protein, acid detergent lignin, extractives, uronic acids, acetyl and so forth.

Additional examples are contemplated where two or more feedstocks are mixed or combined in a range of proportions. For example, in some embodiments, a first feedstock is combined with a secondary feedstock in any suitable proportions as discussed above; in other embodiments a first feedstock and secondary feedstock combination is combined with a tertiary feedstock in any suitable proportions; in other embodiments, the combination comprised of a first feedstock, a secondary feedstock, and a tertiary feedstock, is combined or mixed with a quaternary feedstock; and in other embodiments, the combined feedstock is mixed or further combined with a N^{th} feedstock, where N is a theoretically infinite number of feedstocks or feedstock combinations. In such combinations, according to some embodiments, each constituent feedstock is combined in equal drymass proportion to each additional constituent feedstock. In other embodiments, form a biomass drymass equivalency perspective, the secondary, tertiary, quaternary and N^{th} feedstocks are mixed with the base or first feedstock in any suitable ratio, including 1:1000, 1:100, 1:10, 1:1 and so forth or visa-versa. In still other embodiments, in terms of the drymass or dry matter percentage of the total combined feedstock, the secondary, tertiary, quaternary and/or N^{th} feedstocks are mixed with the base or first feedstock in as any percentage, including where the first feedstock comprises from one one-hundreth to ten percent (0.01%-10%), ten to twenty percent (10%-20%), twenty to thirty percent (20%-30%), thirty to forty percent (30%-40%), forty to fifty percent (40%-50%), fifty to sixty percent (50%-60%), sixty to seventy percent (60%-70%), seventy to eighty percent (70%-80%), eighty to ninety percent (80%-90%), and/or ninety to ninety-nine and ninety-nine one-hundredth percent (90%-99.99%) of the total dry matter while the secondary, tertiary, quaternary and/or N^{th} feedstocks comprise the remaining percentage of the total dry matter.

In some embodiments, as referenced above, algae constitutes the first or based feedstock and acts as a binder or a catalyst suitable for facilitating the post-development processing or refinement of the feedstock mixture. In other embodiments, the appropriate combinations of feedstock constituents is dictated by the combination which optimized or otherwise raises the net energy potential or value of the feedstock combination relative to the energy potential or value of its constituent elements when separate.

In some embodiments, any of the combinations may also be mixed with inorganic components.

### Formatting Biomass Feedstocks

As a corollary to manipulating the composition of a feedstock composition, further embodiments relate to formatting such feedstocks and/or feedstock compositions. According to some embodiments, the feedstock composition is formatted simultaneously with fostering, nurturing, cultivating, collecting, maturing or otherwise growing the feedstock and/or creating appropriate feedstock compositions via blending. In other embodiments, however, the feedstock composition is formatted following the growth and/or blending thereof. In some embodiments, formatting the feedstock comprises combining feedstocks to form an appropriate feedstock composition tailored to facilitate and optimize a particular process. In other embodiments, formatting the feedstock also or alternatively comprises forming or otherwise processing the feedstock or feedstock composition such that it assumes a particular morphology adapted to the subsequent uses, applications, processes, technologies and/or purposes of the composition. For example, in some embodiments, a given feedstock composition is formatted as a liquid, a gas, a powder, a dust, a residue, a concentrate, a briquette, a pellet or a tablet among other suitable formats. In other embodiments, a given feedstock composition is formatted as a combustible or in a combustible form, such as a dry form. In various embodiments, the proper format for a given feedstock composition is correlated with the moisture content of the composition. For example, a liquid format corresponds with a composition having a high moisture content while a powder corresponds with a composition having a low moisture content.

In various embodiments, any appropriate method common to those of skill in the art may be used and executed to format the relevant feedstock composition appropriately. For example, where a feedstock composition is to be formatted as a pellet or a briquette, the briquette may be formed by first creating the relevant composition and then extruding an appropriately dimensioned pellet or briquette. In the various embodiments, the resulting feedstock is suitably formatted for immediate and/or direct use in various applications, processes or technologies. Alternatively, various feedstocks are formatted to facilitate further downstream refinements in some embodiments.

### Refining Biomass Feedstocks

As mentioned above, according to some embodiments, formatted feedstocks are useful for carrying out or facilitating one or more subsequent processes, applications or technologies for producing a spectrum of bio-based products which can be used as substitutes for fossil oils and fossil oil derivatives in product manufacturing processes and/or the production of bio-energy. In other words, according to some embodiments, certain feedstocks or feedstock compositions having a particular format are suitable for subsequently refining or processing the same to derive useful bio-based products or bio-energy therefrom. Some further embodiments contemplate the standardization of certain feedstocks and associated parameters, such as blend or composition, moister content, format, and/or other properties and characteristics of such feedstocks, so as to facilitate large-scale processes, applications or technologies in order to optimize and efficiently scale-up the production of a spectrum of useful bio-based products.

According to some embodiments, for example, various formatted feedstocks are used in, refined through, or otherwise processed in one or more subsequent processes, applications or technologies or subject to one or more subsequent refinement processes such as anaerobic digestion, biochemical fractionation including dry fractionation or solvent fractionation, fermentation, gasification, transesterification, upgrading including hydrothermal upgrading, pyrolysis, torrefaction, hydrotreating including catalytic hydrotreating, Fischer-Tropsch synthesis, hydroforming, enzyme hydrolysis, hydrocracking, co-firing, and other processes familiar to those of skill in the art. In further embodiments, the present invention further relates to systems and methods for identifying and measuring key parameters of the foregoing processes in order to optimize the same.

In yet further embodiments, the foregoing processes are standardized along with the standardization of associated feedstocks, respectively, (along with the standardization of other associated parameters) so as to facilitate scaling such processes, applications or technologies up and to optimize the same. By way of example, several such processes and associated standardizations are discussed in greater detail below. Such examples are given for purposes of illustrating the principles previously discussed and are not intended to be limiting in any respect. Specifically, processes not expressly discussed below but nevertheless falling within the scope of the appended claims are intended to be covered by the same.

### Anaerobic Digestion

Anaerobic digestion is a series of processes in which microorganisms break down biodegradable material in the absence of oxygen, used for industrial or domestic purposes to manage waste and/or to release energy. According to some embodiments, anaerobic digestion is useful as a renewable energy source or process because the process produces a methane and carbon dioxide rich biogas suitable for energy production, helping to replace fossil fuels. In some further embodiments, the nutrient-rich digestate, which is also produced in tandem with the biogas, can be used as fertilizer.

According to some embodiments, anaerobic digestion produces methane from algae. In such embodiments, the process for obtaining methane from algae involves the following successive stages: 1) pre-treatment of the algae, capable of producing a liquid suspension of fine solid particles, said treatment being moreover capable of partially depolymerizing the solid algae matter, 2) running the suspension through a fluidized bed containing granules on which enzymes are immobilized which are capable of transforming the particles into sugar, said liquid containing acidic bacteria capable of transforming said sugars into volatile fatty acids, 3) decantation of the suspension, so as to remove any solid particles that may remain, and to extract a decanted liquid, and 4) running the decanted liquid across a fixed bed containing methanogenic bacteria set onto a support so as to cause the liquid to release a gas mixture containing mainly methane.

In some embodiments, the digestion process begins with bacterialhydrolysis of the input materials in order to break down insoluble organic polymers such as carbohydrates and make them available for other bacteria. In such embodiments, acidogenic bacteria then convert the sugars and amino acids into carbon dioxide, hydrogen, ammonia, and organic acids. Further, according to such embodiments, acetogenic bacteria then convert these resulting organic acids into acetic acid, along with additional ammonia, hydrogen, and carbon dioxide. Finally, methanogens convert these products to methane and carbon dioxide according to the embodiments just described.

### Biochemical Fractionation

Dry fractionation of oils and fats is the separation of high-melting triglycerides, olein, from low-melting triglycerides, stearin, by crystallization from the melt. According to some embodiments, apart from blending, dry fractionation is a relatively inexpensive process in oils and fats processing. It is a pure physical process compared to other chemical modification processes such as hydrogenation and interesterification which modify triglycerides. According to some embodiments, a dry fractionation plant consists of crystallization section and filtration section.

In such embodiments, in the crystallization section, preheated palm oil is fed into the crystallizers and then cooled in a controlled environment to form crystals. According to such embodiments, the cooling sequence follows a defined program using programmable controllers. Further pursuant to such embodiments, the slurry of crystals and oil is then pumped to the fractionation filter for separation of the solid crystals from the oil. In such embodiments, the filtration section, an automated membrane filter press, is used for filtration of oil slurry to separate the stearin crystals from the liquid olein. According to some embodiments, stearin is retained as filter cake while olein passes through the filter as filtrate. In some embodiments, olein yield is maximized by squeezing the stearin cake through inflation of the membrane with air or liquid. Dry fractionation generally only requires crystallizers and filters. Moreover, dry fractionation is non-energy-intensive, which is advantageous with respect to operating costs

### Fermentation

As biomass is a natural material, many highly efficient biochemical processes have developed in nature to break down the molecules of which biomass is composed, and many of these biochemical conversion processes can be harnessed according to various embodiments of the present invention. In some embodiments, for example, biochemical conversions make use of the enzymes of bacteria and other micro-organisms to break down biomass. In such embodiments, micro-organisms are used to perform the conversion process: anaerobic digestion, fermentation and composting. According to such embodiments, fermentation is a series of chemical reactions that convert sugars to ethanol. The fermentation reaction, according to various embodiments, is caused by yeast or bacteria, which feed on the sugars. In some embodiments, ethanol and carbon dioxide are produced as the sugar is consumed. The simplified fermentation reaction equation for the 6-carbon sugar, glucose, is:

C₆H₁₂O₆->2CH₃CH₂OH+2CO₂

As mentioned above, lignocellulosic biomass is the generic term for plant biomass comprising a mixture of sugar polymers (cellulose and hemicellulose) and the aromatic polymer lignin. Cellulose is made up of the C₆ sugar glucose, while hemicellulose consists of a mix of different C₆ and C₅ sugars. In various embodiments, ethanol is obtained through the fermentation of monosaccharides. According to some embodiments, since sugar only occurs as polymers in lignocellulosic biomass, these must be converted to monosaccharides using suitable enzymes (biocatalysts) before ethanol fermentation takes place. Thus, pursuant to such embodiments, in the resulting sugar solution, lignin is the only solid residue present. According to various embodiments, the lignin can be separated from the monosaccharide solution and used to produce energy or other materials and the sugar solution is then fermented to obtain ethanol.

### Gasification

According to some embodiments, biomass gasification is a high-temperature process (600 to 1000° C.) to decompose the complex hydrocarbons of biomass into simpler gaseous molecules, primarily hydrogen, carbon monoxide, and carbon dioxide. In some embodiments, some char and tars are also formed, along with methane, water, and other constituents. In such embodiments, hydrogen and carbon monoxide are the desired product gases, because unlike combustion gases, they can be directly fired into a gas turbine for power generation or used in chemical synthesis. A primary advantage of embodiments contemplating biomass gasification over biomass combustion is that the power generation efficiency of a gas turbine combined cycle system can be as much as twice the efficiency of biomass combustion processes, which uses a steam cycle alone.

Biomass fuels, such as firewood and agriculture-generated residues and wastes, are generally organic. They contain carbon, hydrogen, and oxygen along with some moisture. Under controlled conditions, which, according to some embodiments, are characterized by low oxygen supply and high temperatures, most biomass materials can be converted into a gaseous fuel known as producer gas or syngas (from synthesis gas or synthetic gas), which consists of carbon monoxide, hydrogen, carbon dioxide, methane and nitrogen. This thermo-chemical conversion of solid biomass into gaseous fuel is called biomass gasification. The producer gas produced according to such embodiments has low a calorific value (1000-1200 Kcal/Nm3), but can be burnt with a high efficiency and a good degree of control without emitting smoke. In some embodiments, each kilogram of air-dry biomass (10% moisture content) yields about 2.5 Nm3 of producer gas. In energy terms, the conversion efficiency of the gasification process is in the range of 60%-70% according to such embodiments. Four types of gasifiers are currently available for commercial use: counter-current fixed bed, co-current fixed bed, fluidized bed and entrained flow. Each such gasifier may be used, as understood by those of skill in the art, consistent with the methods and systems of the present invention.

### Transesterification

The process of converting vegetable & plant oils into biodiesel fuel is called transesterification. Transesterification refers to a reaction between an ester of one alcohol and a second alcohol to form an ester of the second alcohol and an alcohol from the original ester, as that of methyl acetate and ethyl alcohol to form ethyl acetate and methyl alcohol. Similar processes are known as interesterification. Chemically, transesterification means taking a triglyceride molecule or a complex fatty acid, neutralizing the free fatty acids, removing the glycerin and creating an alcohol ester. According to some embodiments, this is accomplished by mixing methanol with sodium hydroxide to make sodium methoxide. In such embodiments, this liquid is then mixed into vegetable oil. The entire mixture, according to some embodiments, is then allowed to settle. In such embodiments, glycerin is left on the bottom and methyl esters, while biodiesel, is left on top. The glycerin can be used to make numerous other products common to those of skill in the art and the methyl esters is washed and filtered according to some embodiments of the instant invention.

In some embodiments, transesterification of algal oil is done with Ethanol and sodium ethanolate serving as the catalyst. According to some embodiments, sodium ethanolate can be produced by reacting ethanol with sodium. Thus, in such embodiments, with sodium ethanolate as the catalyst, ethanol is reacted with the algal oil (the triglyceride) to produce bio-diesel & glycerol. The end products of the reaction according to the foregoing embodiments are hence biodiesel, sodium ethanolate and glycerol. In some further embodiments, this end-mixture is separated as follows: ether and salt water are added to the mixture and mixed well; after sometime, the entire mixture separates into two layers, with the bottom layer containing a mixture of ether and biodiesel; this layer is separated; biodiesel is in turn separated from the ether by a vaporizer under a high vacuum; and, as the ether vaporizes first, the biodiesel will remain.

### Hydrothermal Upgrading

According to some embodiments, hydrothermal upgrading (HTU) is a biofuel conversion technology that is especially suitable for wet biomass feedstocks, such as beet pulp, sludge or algae. In such embodiments, at a temperature of 300-350° C. and high pressure, the biomass is converted to a heavy organic liquid containing a mixture of hydrocarbons, which is called “biocrude”—a substance made from the complete biomass including oils and akin to crude oil. According to some embodiments, after processing, using a refinery technology called catalytical hydro-de-oxygenation, a liquid biofuel can be produced that is similar to fossil diesel. According to such embodiments, the liquid biofuel can be blended with fossil diesel in any proportion without the necessity of engine or infrastructure modifications.

A hydrothermal process is one that involves water at elevated temperatures and pressures. High-temperature water (HTW), according to some embodiments, refers to water in its liquid state below its critical temperature and pressure (374° C., 221 bar), whereas it becomes a highly compressible fluid called supercritical water (SCW) above this point. According to some embodiments, an advantage of hydrothermal processing for biomass is that hot water can serve as a solvent, a reactant, and even a catalyst or catalyst precursor. While many biomass compounds (e.g., lignin, cellulose) are not water-soluble at ambient conditions, most are readily solubilized in HTW or SCW. According to some embodiments, these soluble components can be subject to hydrolytic attack, engendering fragmentation of bio-macromolecules. In such embodiments, water, both in its dissociated and native form, can help catalyze hydrolysis and other reactions. According to such embodiments, under mild conditions (250-350° C., 40-165 bar), bio-macromolecules hydrolyze and react in a process called hydrothermal liquefaction, yielding a viscous bio-crude oil.

According to some embodiments, hydrothermal processing obviates the need for feedstock dewatering and drying. For example, according to some embodiments, SCW of biomass with at least 30% moisture requires less energy than drying, mainly because hydrothermal processing avoids the energy penalty associated with the phase change from liquid to vapor. Energy efficiency is typically high for hydrothermal processes according to various embodiments. In some embodiments, hydrothermal upgrading (HTU), a commercial-scale process for hydrothermal liquefaction, achieves 75% thermal efficiency and requires just 2% of the input material's energy content to meet its heat demands. Compared to pyrolysis (discussed below), hydrothermal liquefaction, according to various embodiments, occurs at less severe temperatures and produces bio-oil with a lower oxygen content, less moisture, and a higher heating value.

In some embodiments, oxygen is removed to facilitate hydrothermal upgrading. In some embodiments, biomass contains 40-45% w (DAF, dry and ash free basis) of oxygen. In such embodiments, oxygen removal increases the heating value and it leads to a product with more hydrocarbon-like properties, ultimately causing it to be immiscible with water. In other embodiments contemplating thermochemical liquefaction, the oxygen is removed as carbon dioxide and water. In some embodiments, the HTU process heats the feedstock in liquid water to temperatures between 300 and 350° C., pressures 100-180 bar and a residence time ranging from five (5) to twenty (20) minutes. In such embodiments, up to eighty-five percent (85%) of the oxygen is removed from the biomass. Further, according to such embodiments, HTU product distribution (mass units DAF) feedstock: biomass 100 products: biocrude 45, CO₂ 23, CO 2 (*), organics dissolved (**) 12, H₂O 18(*) includes minor amounts of CH₄ and H₂ (**) light organics such as acetic acid, ethanol.

According to some embodiments, the HTU process can compete with premium diesel made from petroleum when crude prices are near US$50/barrel and biomass can be obtained for US$2.50/GJ. In embodiments contemplating the coupling of a bio-refinery concept with protein extraction, HDO upgrading, and gasification, the products that can be produced expand beyond the biocrude and energy production to include premium cattle fodder, green kerosene for aviation fuel, and naphtha feedstocks for chemical plants.

### Pyrolysis

According to some embodiments pyrolysis and gasification are similar processes of heating with limited oxygen. However, in some embodiments, pyrolysis for liquefaction uses no oxygen while gasification uses a small, controlled amount.

Pyrolysis is the thermal decomposition of biomass occurring, according to some embodiments in the absence of oxygen. It is the fundamental chemical reaction that is the precursor of both the combustion and gasification processes and occurs naturally in the first two seconds according to some embodiments. In some embodiments, the products of biomass pyrolysis include biochar, bio-oil and gases including methane, hydrogen, carbon monoxide, and carbon dioxide. According to some embodiments, depending on the thermal environment and the final temperature, pyrolysis will yield mainly biochar at low temperatures, less than 450° C., when the heating rate is quite slow, and mainly gases at high temperatures, greater than 800° C., with rapid heating rates. In some embodiments, at an intermediate temperature and under relatively high heating rates, the main product is bio-oil.

In some embodiments, pyrolysis processes can be categorized as slow pyrolysis while in other embodiments' pyrolysis processes can be categorized as fast pyrolysis. Slow pyrolysis, according to such embodiments, takes several hours to complete and results in biochar as the main product. On the other hand, embodiments contemplating fast pyrolysis yields 60% bio-oil and takes seconds for complete pyrolysis. In addition, in such embodiments, fast pyrolysis gives 20% biochar and 20% syngas. Further, according to other embodiments, fast pyrolysis processes include open-core fixed bed pyrolysis, ablative fast pyrolysis, cyclonic fast pyrolysis, and rotating core fast pyrolysis systems. According to some embodiments, the essential features of a fast pyrolysis process are: very high heating and heat transfer rates, which require a finely ground feed, are fully controlled reaction temperature of around 500° C. in the vapour phase, residence time of pyrolysis vapours in the reactor less than 1 second, and quenching (rapid cooling) of the pyrolysis vapours to give the bio-oil product.

According to some embodiments, pyrolysis oil can be used directly as a fuel or as an intermediate for production of chemicals. In some embodiments, yields of liquid products as high as seventy-nine (79%) of the initial dry weight of the biomass can be achieved. The process, according to some embodiments, produces no waste and either the pyrolysis gas or charcoal is used to heat the reactor while the other can be used to supplement heating, dry the feedstock, or the charcoal can be sold as a byproduct or the pyrolysis gas can be used to fuel a gas engine. According to some embodiments, pyrolysis oil is greenhouse gas neutral, does not produce SO₂ (sulfur dioxide) produces approximately half of the NO₂ (nitrogen oxide) emissions compared to fossil fuels. In some embodiments, pyrolysis is used for the production of chemicals and while in other embodiments it is used for the production of liquid fuels.

According to various embodiments, a wide range of biomass feedstocks can be used in pyrolysis processes. In such embodiments, however, the pyrolysis process is very dependent on the moisture content of the feedstock, which should be around ten percent (10%). According to some embodiments, at higher moisture contents, high levels of water are produced while at lower levels of water there is a risk that the process only produces dust instead of oil. In other embodiments, on the other hand, high-moisture waste streams, such as sludge and meat processing wastes, require drying before subjecting to pyrolysis.

According to various embodiments, the efficiency and nature of the pyrolysis process is dependent on the particle size of feedstocks. In some embodiments, pyrolysis technologies can only process small particles to a maximum of 2 mm keeping in view the need for rapid heat transfer through the particle. Thus, according to such embodiments, the demand for small particle size means that the feedstock has to be size-reduced before being used for pyrolysis.

### Torrefaction

According to some embodiments, torrefaction is a thermo chemical treatment of biomass at 200 to 320° C. In such embodiments, torrefaction is carried out under atmospheric conditions and in the absence of oxygen. During the process, according to some embodiments, the water contained in the biomass as well as superfluous volatiles are removed, and the biopolymers (cellulose, hemicellulose and lignin) partly decompose giving off various types of volatiles. The final product of the forgoing embodiments is the remaining solid, dry, blackened material which is referred to as “torrefied biomass” or “bio-coal.”

During the process, according to some embodiments, the biomass loses twenty percent (20%) of its mass (dry bone basis), while only ten percent (10%) of the energy content in the biomass is lost. According to such embodiments, this energy (the volatiles) can be used as a heating fuel for the torrefaction process. Further, according to such embodiments, after the biomass is torrefied it can be densified, usually into briquettes or pellets using conventional densification equipment, to further increase the density of the material and to improve its hydrophobic properties

According to some embodiments, torrefied and densified biomass has several advantages in different markets, which makes it a competitive option compared to conventional biomass (wood) pellets: energy density of 18-20 GJ/m3 compared to 10-11 GJ/m3 driving a 40-50% reduction in transportation costs.

According to some embodiments, torrefied biomass can be produced from a wide variety of raw biomass feedstocks while yielding similar product properties. According to such embodiments, this is due to the lignocellulose common to many biomass polymers. As discussed previously, in general, (woody and herbaceous) biomass consists of three main polymeric structures: cellulose, hemicellulose and lignin. Together these are called lignocellulose. According to various embodiments, the chemical changes of these polymers during torrefaction are practically similar resulting in similar property changes.

According to some embodiments, torrefied biomass has hydrophobic properties, and, when combined with densification, makes bulk storage in open air feasible. According to some embodiments, torrefaction of biomass leads to improved grindability of biomass. In further embodiments, this leads to more efficient co-firing in existing coal fired power stations or entrained-flow gasification for the production of chemicals and transportation fuels.

### Hydrotreating, Catalytic Hydrotreating

According to some embodiments, the hydrotreating process, a petroleum refining process employed in petroleum refineries, can convert the triglycerides derived from the algae into n-alkanes in a more efficient and economical way. According to various embodiments, the triglyceride reacts with hydrogen at high temperature and pressure over a catalyst in one processing step. According to such embodiments, the products include the straight chain alkanes, CO, CO₂, water, methane, and propane. After a series of separations, according to further embodiments, the primary product is a mixture of straight chain alkanes with carbon numbers ranging from C₁₃ to C₂₀ (C₁₃H₂₈ to C₂₀H₄₂). These n-alkanes are suitable for direct blending into a diesel pool or for further upgrading/reforming into gasoline, jet fuel, or gasoline according to some embodiments.

In some embodiments, the hydrotreating process can be divided into the following components: 1) preparation of triglyceride and hydrogen feed, 2) hydrotreating reactor, 3) stream separations, 4) product separations, and 5) gas scrubbing and recycle. According to some embodiments, a triglyceride feedstock is used instead of crude oil. While triglyceride feedstock can be run through existing hydrotreating units, some adjustments in the design, according to some embodiments, are made to account for the properties of lipid feedstock. In such embodiments, these adjustments include additional quench zones in the hydrotreating reactor to account for the exothermic reactions and modifications to the makeup gas and recycle gas streams.

According to some embodiments, the following table, Table 1, identifies and summarizes several illustrative processes, as described in greater detail above, and associated categorical standards for suitable feedstocks and other associated parameters in order to process biomass so as to derive bio-based products according to the methods of the present invention. The following table is for illustrative purposes and is not intended to be limiting.

TABLE 1
PROCESS COMPARISON - TYPICAL CONDITIONS

| | | | | | HYDROTHERMAL |
|---|---|---|---|---|---|
| PROCESS | ANAEROBIC | BIOCHEMICAL | | | UPGRADING- |
| PARAMETER | DIGESTER | FRACTIONATION | FERMENTATION | GASIFICATION | LIQUEFACTION |
| FEEDSTOCK | Wet Biomass | Lipid Triglycerides | Wet Biomass | Air Dry Biomass 10% | Wet Biomass |
| | | | | Water | 30% Water |
| ADDITIVES | Various bacteria | None | Various bacteria | None | None |
| MAJOR PRODUCT | Biogas | Feedstocks for other | Biogas | Syngas | BioCrude |
| | Methane Carbon | separation processes | Ethanol | | |
| | Dioxide | | | | |
| OTHER | Fertilizer Waste | None | Fertilizer | Char & Tar | Carbon Dioxide |
| BYPRODUCTS | Water | | Waste Water | | Water |
| TYPE OF | Hydrolysis | Melting & | | Thermo-chemical | Removal of Oxygen |
| REACTION | xxxgenesis | Crystallization | | | Hydrolysis |
| TEMPERATURE | 30-60 C. | | | 600-1000 C. | 300-350 C. |
| PRESSURE | Minimal | | | | 100-180 bar |
| EFFICIENCY | | | | 60-70% | 75% |
| | | | | | CATALYTIC |
| PROCESS | | | TRANSESTER- | HYDROTHERMAL | HYDROTHERMAL |
| PARAMETER | HYDROTREATING | PYROLYSIS | IFCATION | CARBONIZATION | GASIFICATION |
| FEEDSTOCK | Lipids | Air Dry Biomass | Lipids | Wet Biomass | 10-20% Wet |
| | | 10% Water | | | Biomass |
| ADDITIVES | None | | Ethanol Sodium | Catalyst | Catalyst |
| | | | Ethanolate | | |
| MAJOR PRODUCT | Biofuels | Bio-oil | Biodiesel | Nano Particles of | Methane |
| | | Metane Hydrogen | Glycerol | BioCoal in Water | Carbon Dioxide |
| OTHER | None | Biochar Carbon | Sodium Ethanolate | Various Stages | Recycle CO2 |
| BYPRODUCTS | | Dioxide | | | |
| TYPE OF | High temperature | Endothermic | | Exothermic | Endothermic |
| REACTION | Cracking | | | | |
| TEMPERATURE | | 400-600 C. | | 250 C. | 300-350 C. |
| PRESSURE | | | | 50 bar | 200 bar |
| EFFICIENCY | | | | 100% carbon eff | |

### Single Step Extraction of Intracellular Products

According to some embodiments, additional systems and methods are contemplated for developing and/or processing certain biomass feedstocks, including lipid feedstocks. For example, in some embodiments, as mentioned above, the present invention relates to extracting intracellular products from microalgae, including lipids, and to the lipid products extracted from these systems and methods. In such embodiments, the systems and methods of the invention can advantageously extract valuable intracellular products from microalgae at a high volume flow rate. By separating non-polar lipids (e.g., triglycerides) from polar lipids (e.g., phospholipids and chlorophyll) and cellular debris, the methods and systems of the invention can produce a product suitable for use in traditional petrochemical processes, such as petrochemical processes that utilize precious metal catalysts. However, in other embodiments, as mentioned above, whole algae feedstocks, with the lipids still housed within the algae cells, are directly used as process feedstocks directed at producing useful bio-products and/or bio-energy without cell disruption or lysing according to and consistent with the methods and systems described previously. Likewise, in yet additional embodiments, algae feedstocks that are only partially lysed or fractured are contemplated.

In embodiments contemplating extraction of the lipids, however, the present invention relates to a method for extracting such lipids from microalgae in a flowing aqueous slurry. In some embodiments, a method is contemplated for doing the same, generally including (i) providing an aqueous slurry including microalgae; (ii) providing a lipid extraction apparatus having a body including a channel that defines a fluid flow path, at least a portion of the channel formed from a cathode and an anode which are spaced apart to form a gap within the channel; (iii) flowing the aqueous slurry through the channel and applying an emf across the gap, the emf compromising the microalgae cells and releasing a lipid; and (iv) recovering at least a portion of the lipid.

In some embodiments, performing the step of providing an aqueous slurry including microalgae comprises providing a microalgae slurry comprised principally of water and algae. In embodiments contemplating the use of an aqueous slurry, the costs normally associated with drying the algae before extraction can be avoided. As mentioned above, in various embodiments, the algae cells can be any microalgae cells, including, but not limited to, Nanochloropsis oculata, Scenedesmus, Chlamydomonas, Chlorella, Spirogyra, Euglena, Prymnesium, Porphyridium, Synechoccus, Cyanobacteria and certain classes of Rhodophyta single celled strains. The algae can be phototrophic bacteria grown in an open natural environment or in a closed environment. The present method can also be used to extract lipids from heterotrophic bacteria.

In various embodiments, the concentration of the algae in the slurry will depend in part on the type of algae, the growth conditions, and whether the algae has been concentrated. The algae can be grown, cultivated and/or used at any suitable concentration. For example, in some embodiments, the algae is allowed to grow naturally in order to mimic or imitate algae concentrations found in nature. In other embodiments, the concentration of algae or microalgae contained in the slurry can be increased or augmented using any known technique. For example, concentrating or increasing the percentage of algae in a given or select volume of water can be carried out using flocculation. The flocculation can be a chemical flocculation, an electro-flocculation or any other process that effectuates a similar agglomeration or coagulation of algae cells.

According to some embodiments, the purity of the slurry with respect to the concentration of microalgae as a percentage of the total microorganisms in the slurry can impact the composition of the lipids released from the extraction process. In such embodiments, the composition of lipids a user desires from the extraction process can dictate an appropriate concentration of microalgae in the slurry thus rendering certain concentrations desirable according to user preferences.

As mentioned above, in some embodiments the concentration of microalgae within a select quantity or volume of microalgae slurry can be increased using, among other techniques, flocculation. In this way, the microalgae cells can be efficiently harvested for subsequent processing, including but not limited to lipid extraction, while minimizing the capture of water associated with the microalgae cells. In other words, according to some embodiments flocculating the algae cells increases the concentration of such cells in a common region or area of the overall slurry thereby permitting the algae cells to be efficiently captured for harvesting by selecting or capturing that portion of the slurry in which the algae cells are most densely concentrated.

According to some embodiments, the microalgae slurry exhibits characteristics consistent with liquid phase hydrocolloidal systems or hydrocolloidal suspensions in which the microalgae constitutes colloids or colloidal particles dispersed throughout the water based slurry. In such embodiments, the behavior of the microalgae colloids adheres to colloidal chemistry principles. As such, zeta potential may be used to monitor the growth and development of the algae and relevant parameters can be adjusted according to zeta potential to encourage the algae to agglomerate. According to some embodiments, it is contemplated that the zeta potential associated with a select microalgae stock are useful for monitoring and ultimately controlling microalgae growth and flocculation. For example, in various embodiments, monitoring the zeta potential permits a user to determine the correct emf or EMP to apply to the slurry to optimize growth and/or flocculation.

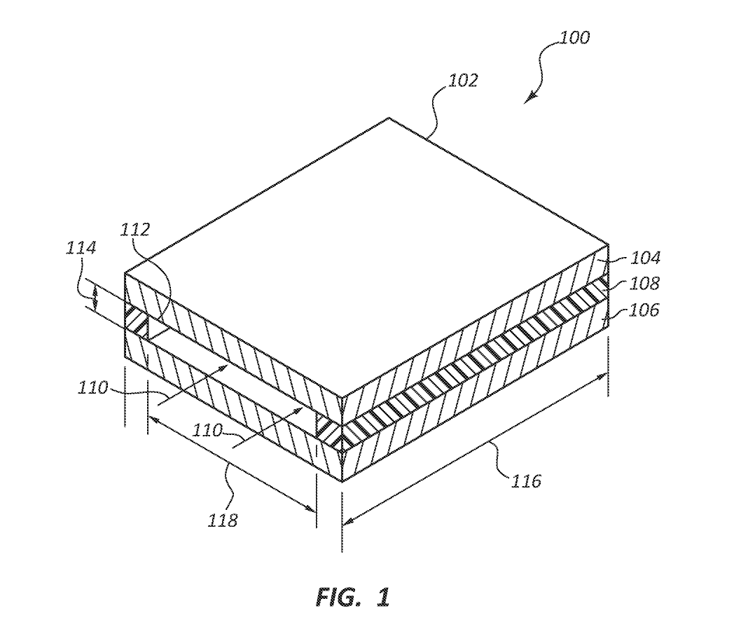

In addition to the step of providing an aqueous slurry including microalgae, which in some embodiments includes optimizing the growth and/or flocculation of the microalgae according to the various embodiments discussed above, another step is contemplated according to some embodiments. Specifically, with reference to FIG. 1, in some embodiments a lipid extraction apparatus 100 is provided that includes an anode 104 and a cathode 106 that form a channel 112 through which the aqueous slurry can flow.

FIG. 1 illustrates a portion of a lipid extraction device 100 according to some embodiments that is suitable for use in the method of the invention. The portion of extraction device 100 includes a body 102 comprised of anode 104 and cathode 106, which are electrically separated by an insulator 108. Anode 104 and cathode 106 are spaced apart to form channel 112, which in turn defines a fluid flow path 110. Channel 112 has a length 116 that extends the length of the anode and the cathode. In some embodiments, the fluid flow path 110 is exposed to both the anode and the cathode along the entire length 116. Channel 112 also has a width 118 that is defined by the space between the insulators 108. In some embodiments, the fluid flow path is exposed to the anode and cathode along the entire width 118. In some embodiments, insulators 118 define a gap 114 between anode 104 and cathode 106.

In various embodiments, gap 114 between anode 104 and cathode 106 has a distance suitable for applying an emf through the aqueous algae slurry. In some embodiments, a narrow gap 114 coupled with a large width 118 and length 116 can provide a large volume for channel 112 while maintaining a strong electrical field for compromising the algae cells to release polar lipids. The length 116 of channel 112 is the dimension in the direction of fluid flow 110 and can be any length so long as channel is not hampered by plugging or significant pressure drops. The width 118 can be any dimension so long as the materials of the anode and cathode are sufficiently strong to span the width without contacting one another.

In various embodiments, the anode 104 and cathode 106 can be made of any electrically conductive material suitable for applying an emf across the gap, including but not limited to metals such a steel and conductive composites or polymers.

The shape of the lipid extraction device, and the anode and cathode constituents, can be planer or cylindrical or any other desirable shape. As described more fully below, an annulus created between an inner metallic surface of a larger external tube and an outer surface of a smaller metallic conductor tube placed within the large tube diminishes or eliminates fouling while maintaining a high surface area in a compact design. The tubes need not have a circular periphery as an inner or outer tube may be square, rectangular, or other shape and the tube shape does not necessarily have to be the same, thereby permitting tube shapes of the inner and outer tubes to be different according to some embodiments. In one embodiment, the inner conductor and outer tube are concentric tubes, with at least one tube being provided with a plurality of spiral grooves separated by lands to impart a rifling to the tube. This rifling has been found to decrease build-up of residue on the tube surfaces. In commercial production, there may be a plurality of inner tubes surrounded by an outer tube to increase the surface contact of the metal conductors with the algae.

In various embodiments, the use of electrical insulators, such as plastic tubes, baffles, and other devices, can be used to separate a large lipid extraction device into a plurality of zones, so as to efficiently scale-up the invention to commercial applications.

In some embodiments, it is contemplated that the aqueous algae slurry is fed through channel 112 along fluid flow path 110 between the anode and cathode (i.e., through gap 114). Power is applied to the anode and cathode to produce an electromotive force that compromises or lyses the algae cells in order to induce the release of intracellular products, such as lipids. For a given gap distance and/or channel volume between the anode and cathode, the amperage, flow rate, and voltage are selected to effectuate the release of intracellular products, such as lipids.

Similar to some of the methods for optimizing and/or controlling the growth and/or flocculation of algae as discussed in detail above, in some embodiments zeta potential is used to optimize and/or control the power applied to the anode and cathode to produce a suitable electromotive force for compromising or lysing the algae cells as they flow through lipid extraction device 100. Specifically, according to some embodiments, monitoring zeta potential permits a user to determine and optimize the emf applied to the slurry. In other words, for a given gap distance and/or channel volume, the amperage, flow rate and voltage can be optimized to maximize the efficiency of inducing the release of intracellular products. In still other embodiments, additional parameters, such as duration, frequency, pulse and the like, can be optimized to increase system efficiencies. In this way, the minimum amount of power input can be identified and applied such that the cells are adequately fractured without the needless expenditure of superfluous energy. In addition, the dimensions of lipid extraction apparatus 100 can be optimized, including the width, length and gap, so as to minimize the size of apparatus 100 thereby conserving resources and space while maximizing the flow rate in order to process the slurry quickly and efficiently.

As above, in some embodiments, an emf can be applied to the slurry manually based on corresponding zeta potential measurements. In other embodiments, an automated system is contemplated in which various components of the system interact to both monitor zeta potential, select the correct emf parameters, and automatically apply an appropriate emf to the slurry to control lysing or fracturing. In some embodiments, zeta potential can be monitored periodically using a zeta potential meter (not shown) common to those of skill in the art. In such embodiments, the zeta potential value can be supplied to the system as it is periodically measured in order to control cell lysing according to zeta potential values. In other embodiments, it is contemplated that zeta potential can be monitored and controlled continuously and/or in real time using a calibrated streaming current device (not shown) common to those of skill in the art. In some embodiments, the streaming current device can be calibrated via a zeta potential meter such that the streaming current device provides measurements in mV. In such embodiments, the zeta potential of the slurry is continuously measured in real time and utilized within the system to control and optimize cell fracturing. In embodiments contemplating a streaming current device, the device can be integrated within the system or the system can comprise such a device such that the device functions as an on-line monitor of zeta potential.

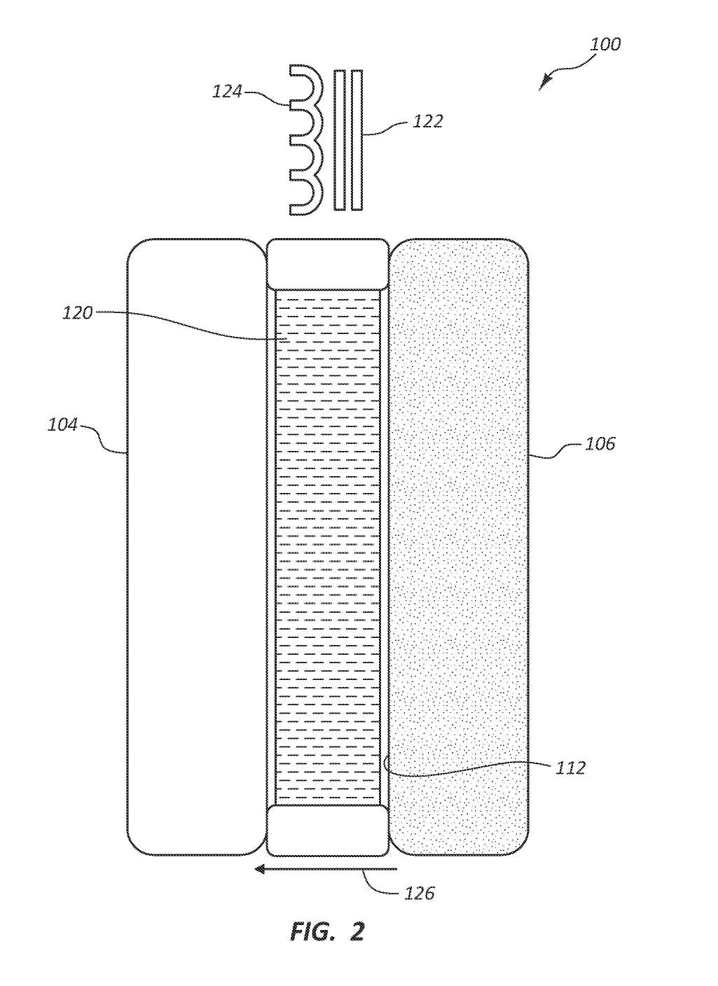

With reference now to FIG. 2, lipid extraction apparatus 100 according to some embodiments is shown in cross section with an aqueous algae slurry 120 disposed between cathode 106 and anode 104. In some embodiments, aqueous algae slurry 120 is caused to flow through channel 112 using a pump (not shown). In other embodiments, the aqueous algae slurry is gravity fed through channel 112. By way of an electrical conduit, a negative connection 122 is made to anode 104, which provides electrical grounding. Positive electrical input 124, also delivered by way of a conduit connection, provides positive electrical transfer throughout cathode 106. When a positive current 124 is applied to cathode 106, the current seeks a grounding circuit for electrical transfer as indicated by arrow 126 or, in the illustrated embodiment, to anode 104, which allows the completion of the electrical circuit. In this respect, transfer of electrons occurs between the positive and negative surfaces areas but only when an electrically conductive liquid is present between them. As the liquid medium containing algae slurry 120 is flowed between the surface areas, electrical transfer from cathode 106 through slurry 120 to anode 104 is made. As a liquid containing a microorganism biomass transverses the anode and cathode circuit, the cells are exposed to the electric field that causes expansion and contraction of the cells.

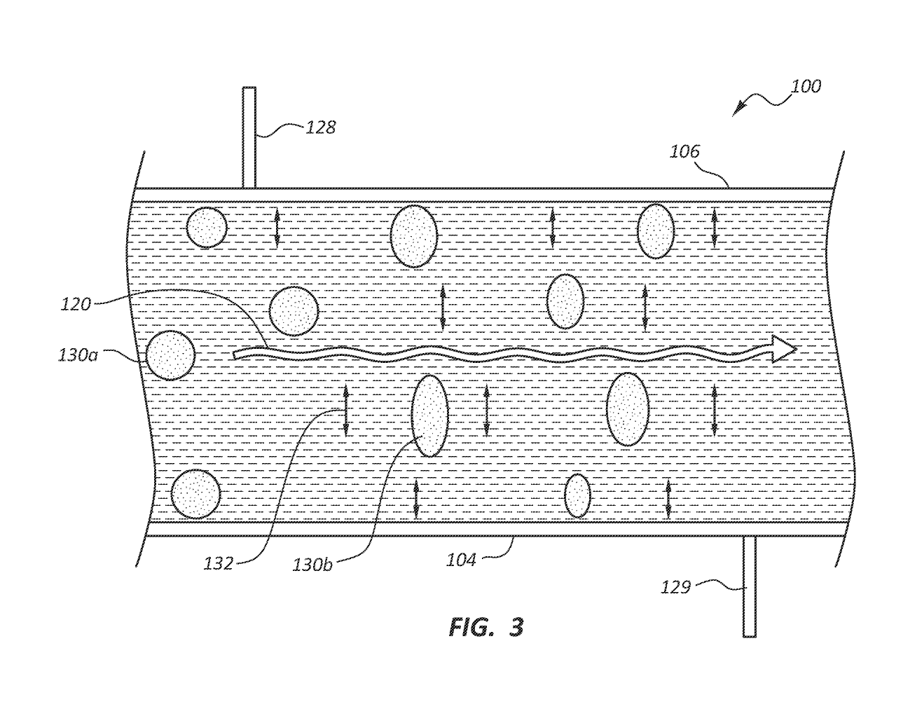

According to some embodiments, FIG. 3 illustrates an emf transfer between two electrically conductive electrodes, such as metallic walls, with a liquid medium containing a living microorganism biomass flowing between them. The illustration depicted in FIG. 3 denotes a method according to some embodiments for fracturing or lysing the algae cells in order to harvest biomass from an aqueous solution containing such cells. As depicted, cathode 106 requires a positive electrical connection point 128, which is used for positive current input. Positive transfer polarizes the entire length and width of cathode 106 and seeks a grounding source in anode 104. In order to complete an electrical circuit, anode 104 includes a grounding connection point 129, which allows an electrical transfer 132 to occur through aqueous slurry 120. The aqueous slurry includes a liquid medium that contains a nutrient source mainly composed of a conductive mineral content that was used during a growth phase of the algae in aqueous slurry 120. The liquid medium containing the nutrient source further allows positive electrical input to transfer between electrodes 104, 106 through the liquid medium/biomass 120, which only occurs when the liquid medium is present or flowing. Electrical input causes cellular elongation such as the distention shown in algae 130b as compared to algae 130a.

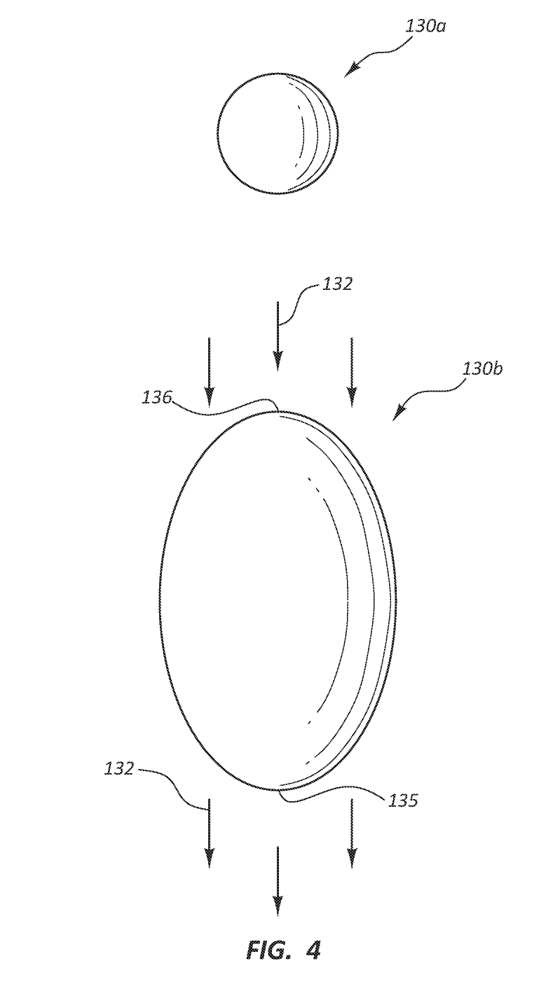

Turning to FIG. 4, an isolated illustration of algae cells 130a and 130b is provided to exhibit the difference between a normal sized microalgae cell (130a) in comparison to a microalgae cell (130b) that has been extended by the electrical field between the cathode and anode. During the electrical on phase, emf 132 polarizes the algae cell walls and/or membranes. The dipole on the cells 135, 136 causes the cells to be pulled apart along the electrical field lines, thereby releasing the cell contents. This elongation eventually causes external structural damage to the exterior wall with general damage resulting to a wall and membrane that is permeable to the intracellular fluids. In other words, the elongation eventually causes cell lysis. In some embodiments, the flow rate, voltage, amperage and/or other parameters are selected in combination with the gap distance and composition of the aqueous slurry to primarily cause the release of non-polar lipids without releasing the polar lipids, including those in the cell membrane, such as chlorophyll and phospholipids. As discussed above, in some embodiments the various parameters are optimized to enhance the efficiency of cell lysis by using zeta potential.

In some embodiments, the flow rate can be controlled by means of a pump (not shown) or other suitable fluid flow mechanical devices. In other embodiments, the flow rate can be controlled by the geometry or design of the system in connection with gravity. In various embodiments, any suitable flow rate can be used. Likewise, in some embodiments, the voltage and/or amperage can be controlled by an adjustable electrical source based on zeta potential, as measured by a zeta potential meter or calibrated streaming current device. In still other embodiments, the voltage and/or amperage can be controlled by an adjustable electrical source based on user experience, post processing data, or other useful and measurable parameters. Again, in various embodiments, any suitable voltage and/or amperage can be used.

In one embodiment, the emf can be pulsed on and off repeatedly to cause recurring extension and relaxation of the algae cells. In this embodiment, voltages can be higher and peak amperage lower while average amperage remains relatively low. Such embodiments reduce the energy necessary for operating the apparatus and minimize wear on the anode and cathode. In such embodiments, the frequency of the emf pulse, among other parameters discussed above, can be controlled by according to zeta potential, as measured by a zeta potential meter or calibrated streaming current device. In the various embodiments, any suitable pulse frequency can be used.

In other embodiments, the temperature of the aqueous slurry during extraction can also have an impact on the power required to extract desirable intracellular products, such as lipids. In some embodiments, intracellular product extraction may be carried out at room temperature. In other embodiments, however, heat is added to the aqueous algae slurry to achieve a desired temperature in order to enhance intracellular product extraction.

In various embodiments, the temperature of the slurry can also be adjusted to control the specific gravity of the water relative to the algae. As the liquid medium (typically mainly composed of water) is heated, alterations to its hydrogen density occur; this alteration of density allows normally less dense material to sink. For example, in some embodiments, the heavier fractured cellular mass and debris material, which would normally float, rapidly sinks to the bottom of the liquid column due to the alteration of the density of the liquid medium. In some embodiments, such alterations also enhance the efficiency and ease of harvesting the cellular mass, which is useful for other product applications. As used in this description “specific gravity” is a dimensionless unit defined as the ratio of density to a specific material as opposed to the density of the water at a specified temperature.

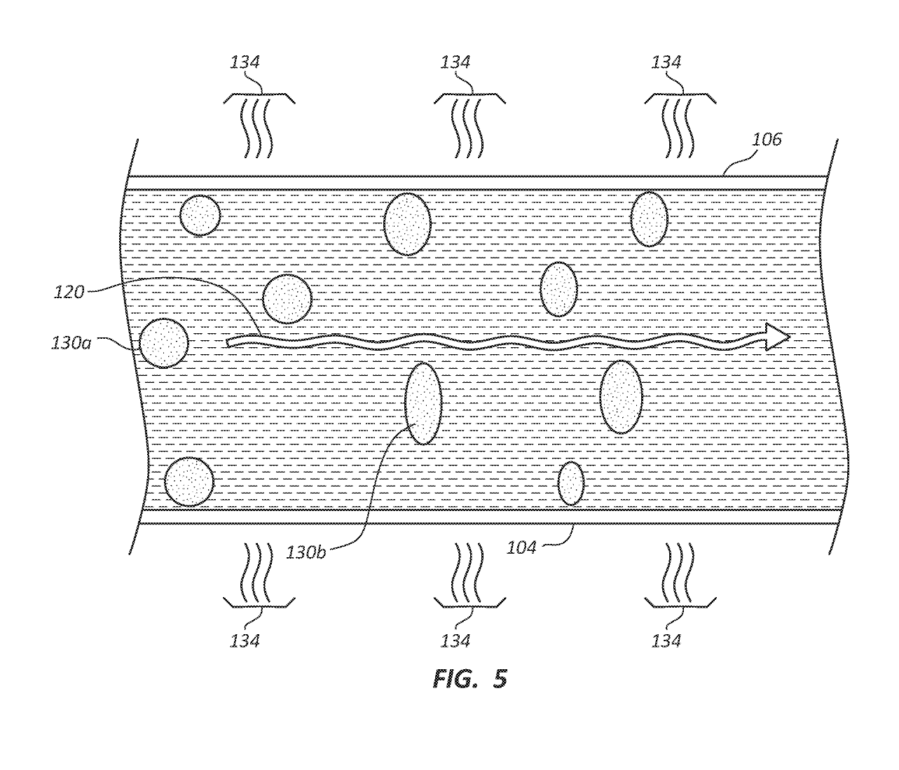

In reference to FIG. 5, a heat transfer according to some embodiments is illustrated. As depicted, in some embodiments a heat transfer can occur through the outer walls of either cathode 106, anode 104 or both such that the liquid medium/biomass can be heated. In some embodiments, the liquid medium can be heated during the emf process while in other embodiments the liquid medium can be heated before and/or after the emf process. As discussed above, by applying heat the cellular mass and debris from an aqueous solution containing algae can be efficiently separated and harvested. In some embodiments, a heating device 134 can be attached to the outside surfaces of either cathode 106, anode 104 or both such that heat transfer is allowed to penetrate into the aqueous slurry 120.

Once the emf (or pulsed emf) and/or heating processes discussed above have been completed according to some embodiments, the liquid medium containing a now fractured biomass is transferred into a secondary holding tank where a liquid pump allows a continuous loop flow. In some embodiments, the secondary holding tank comprises a clarifier, such as a gravity clarifier, wherein the intracellular products, such as desirable lipids, are allowed to float to the top and be collected, the biomass sinks to the bottom such that it can be harvested and the water may be recycled and reused to facilitate the growth and/or processing of subsequent algae stocks.

The products recovered from the methods of the present invention can have a relatively low content of polar lipids such as chlorophyll and phospholipids while having a relatively high content of non-polar lipids.

The methods of the invention may further include reducing the content of phosphorus and using the non-polar lipids in at least one catalytic refining process. For example, the lipids can be hydrotreated using a supported catalyst.

In some embodiments, it is contemplated that a portion of algae can be periodically drawn out from a growing algae source or stock and processed to extract intracellular products as discussed above while maintaining a steady rate of growth. Steady state growth can be achieved by drawing algae at a rate of less than half the algae mass per unit time that it takes for the algae to double. In one embodiment, algae is harvested at least as often as the doubling time of the algae, more preferably at least twice during the doubling time of the algae. The doubling time will depend on the algae type and growth conditions but can be as little as 6 hours to several days. In some embodiments, however, the growth conditions can be modified through monitoring zeta potential and adjusting pH and/or providing nutrients, such as CO₂, during the growth phase.

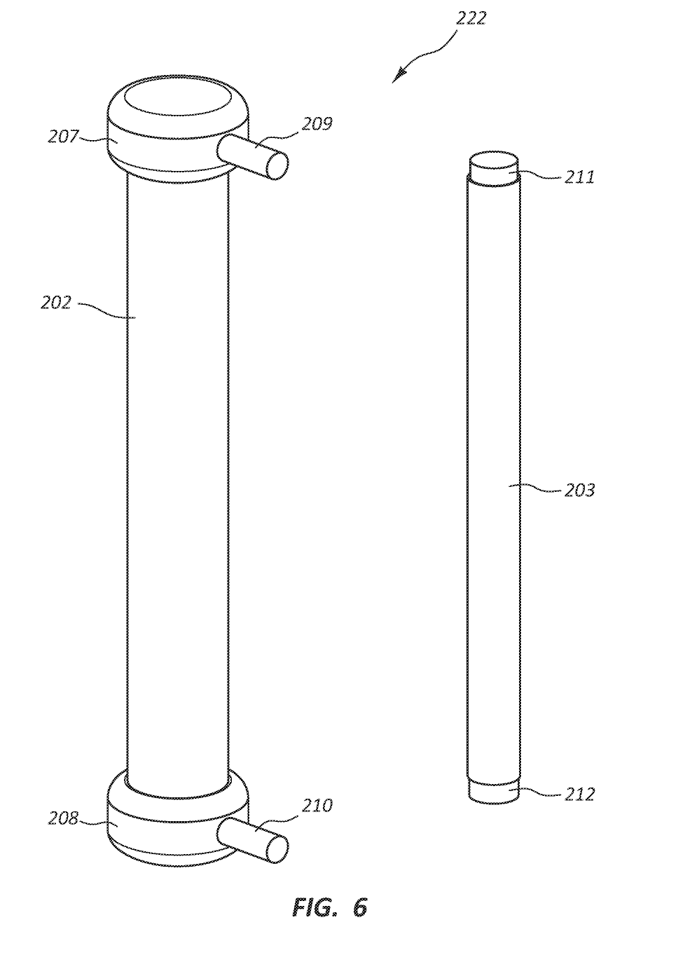

With reference now to FIGS. 6 through 8, various examples of how lipid extraction apparatus 100 can be implemented are described. In some embodiments, for example, an embodiment of apparatus 100 is depicted at 222 as a “tube within a tube” configuration. The apparatus 222 illustrated in FIG. 6 is shown in a disassembled configuration for the convenience of explaining its constituent elements according to some embodiments. Specifically, in some embodiments, lipid extraction device 222 comprises a first conductive tube 203 (hereinafter “cathode 203”, although conductive tube 203 may also be the anode or switch between anode and cathode) and a second conductive tube 202 (hereinafter “anode 202”, although conductive tube 202 may also be the cathode or switch between anode and cathode). In such embodiments, cathode 203 is configured to be placed within anode 202.

In some embodiments, anode 202 includes a pair of containment sealing end caps 207 and 208. In such embodiments, sealing end cap 207 provides an entry point 209 used to accept an aqueous algae slurry. Likewise, after biomass transiting, the opposing end cap 208 provides an exit point 210 to the outward flowing algae biomass. As further depicted in FIG. 6, cathode 203 also includes sealed end caps 211 and 212 to prevent liquid flow through the center of the tube and to divert the flow between the inner surface of anode 202 and the outer surface of cathode 203, thereby forming a channel. The channel can be sized and configured as described above with respect to FIG. 1. The use of a “tube within a tube” configuration militates against fouling by the algae and/or other organisms in the slurry.

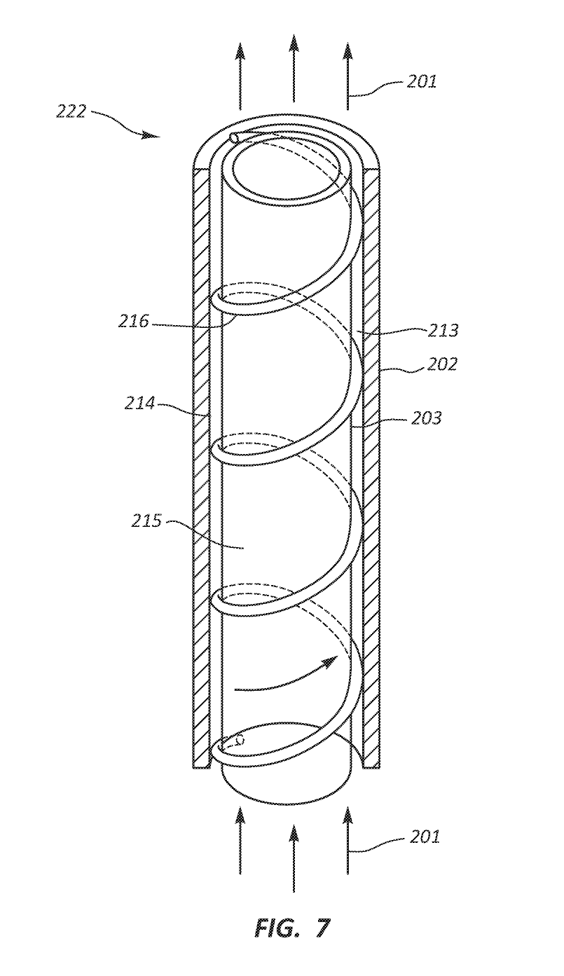

With reference to FIG. 7, another embodiment of apparatus 100 is depicted at 222. As shown, in some embodiments an insulative spacer 213 is positioned in the channel defined between anode 202 and cathode 203. In one embodiment, the insulative spacer forms a helix or coil to cause spiraling fluid flow. In other embodiments, insulative spacer(s) 213 can be straight or curved in any manner so long as they do not occlude the channel between anode 202 and cathode 203. In various embodiments, insulative spacer 213 serves as a liquid seal between the two wall surfaces 214 and 215 with the thickness of the spacer preferably providing equal distance spacing between anode 202 and cathode 203. In some embodiments, spacer 213 prevents contact between anode 202 and cathode 203, which prevents shorting and forces electrical current through the liquid medium. Further the insulator 213 provides a gap 216 between the two wall surfaces 214 and 215 allowing a passage way for a flowing biomass 201. Any suitable material can be used as a spacer. Typically, ceramic, polymeric, vinyl, PVC plastics, bio-plastics, vinyl, monofilament, vinyl rubber, synthetic rubber, or other non-conductive materials are used.

In embodiments contemplating a helical or spiraling insulative spacer 213, the spacing and directional flow can cause the fluid flow path to complete a three hundred and sixty degree transfer of electrical current around anode 202 and cathode 203. The spiraling directional flow further provides a longer transit duration which provides greater electrical exposure to the flowing biomass 201 thus increasing substance extraction efficiency at a lower per kilowatt hour consumption rate during intracellular substance extraction.

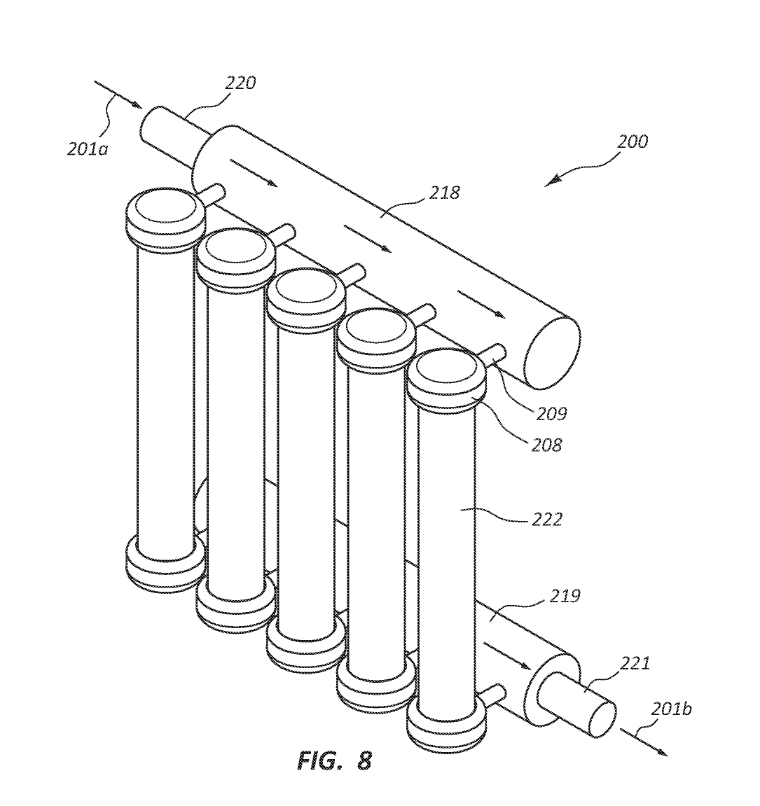

Turning to FIG. 8, a series of anode and cathode circuits 222 are shown in parallel according to some embodiments. In such embodiments, the series of electrode circuits 222 combine to form a lipid extraction apparatus or system 200. As depicted, some embodiments of system 200 comprise a common upper manifold chamber 218, which receives an in flowing biomass 201a through entry port 220. Once entering into the upper manifold chamber 218, the biomass 201 flows downward into the individual anode and cathode circuits 222 through entry ports 209, which allow a fluid connection to the sealing end caps 208. In such embodiments, the flowing biomass 201 (i.e., the aqueous algae slurry) is subjected to an emf or EMP within the anode and cathode circuits 222 in order to fracture the algae cells in accordance with various embodiments of the invention. As discussed above, in some embodiments, the emf or EMP is controlled according to zeta potential. Further, according to some embodiments, zeta potential is being measured continuously in order to control the emf or EMP in real time as the flowing biomass 201 traverses circuits 222. Once transiting through the individual circuits 222, the flowing biomass 201 exits into a lower manifold chamber 219 where the biomass 201b is then directed to flow out of the system 200 through exit point 221.

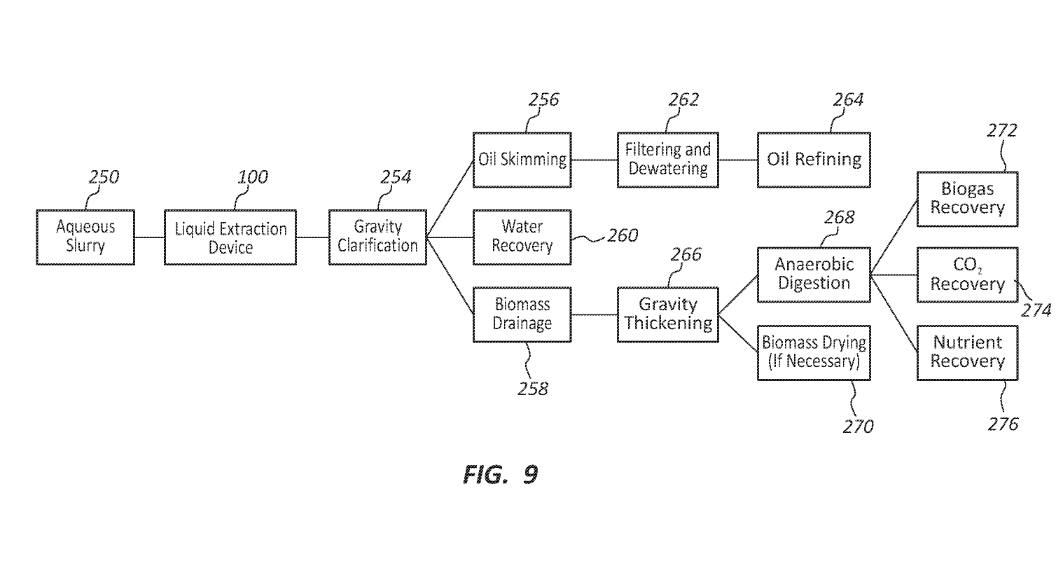

With Reference to FIG. 9, an overall process is described for growing and subsequently processing an algae slurry and extracting intracellular products, such as lipids, therefrom. In various embodiments, the methods, systems, and/or apparatuses disclosed herein can use all or a portion of the steps and/or apparatuses shown in FIG. 9.

In some embodiments, a method is contemplated for harvesting at least one intracellular product from algae cells in aqueous suspension. In such embodiments, the algae stock, comprised of algae cells, is grown in a growth chamber or reactor 250. In various embodiment, growth chamber or reactor 250 can comprise any body of water, container or vessel in which all requirements for sustaining life of the algae cells are provided for. Examples of growth chambers 250 include, but are not limited to, an open pond or an enclosed growth tank. In some embodiments, growth chamber 250 is in fluid communication with an extraction apparatus 100 (or 200 or 222) as described herein such that algae cells within growth chamber 250 can be transferred to apparatus 100 (e.g., by way of gravity or a liquid pump). In such embodiments, the living bio mass is flowed via a conduit into the inlet section of the anode and cathode circuit as described above. In various embodiments, the algae slurry within growth chamber 250 can be transferred to apparatus 100 by any suitable device or apparatus, e.g., pipes, canals, or other conventional water moving apparatuses. In order to harvest at least one intracellular product from the algae cells in accordance with some embodiments, the algae cells are moved from growth chamber 250 to an apparatus 100 (or other apparatus as described above with reference to FIGS. 1-8) and contained within apparatus 100. When added to the apparatus 100, the algae cells are generally in the form of a live slurry (also referred to herein as “biomass”). The live slurry is an aqueous suspension that includes algae cells, water and nutrients such as an algal culture formula based on Guillard's 1975 F/2 algae food formula that provides nitrogen, vitamins and essential trace minerals for improved growth rates in freshwater and marine algae. Any suitable concentration of algae cells and sodium chloride, fresh, brackish or waste water can be used, such that the algae cells grow in the aqueous suspension.

According to some embodiments, after the intracellular products are released from the fractured algae cells in apparatus 100, the slurry (comprised of intracellular products 256 as well as fractured cellular mass and debris 258) may be subjected to one or more downstream treatments including gravity clarification. Gravity clarification generally occurs in a clarification tank 254 in which the intracellular product(s) of interest 256 (e.g., lipids) rise to the top of tank 254, and the cellular mass and debris 258 sinks to the bottom of tank 254. In such an embodiment, upon traversing the extraction circuit apparatus 100, the slurry (comprised of intracellular products 256 as well as fractured cellular mass and debris 258) is flowed over into gravity clarification tank 254 that is in fluid communication with apparatus 100 in order to facilitate the separation of cellular mass and debris 258 from intracellular products 256 from algae cells as described herein. In gravity clarification tank 254, the lighter, less dense substances (e.g., lipids) float to the top of the liquid column while the heavier, denser materials (e.g. cellular mass and debris) sink to the bottom for additional substance harvest.

In such embodiments, the intracellular product(s) of interest 256 are then easily harvested from the top of clarifier 254 such as by skimming or passing over a weir, and the cellular mass and debris 258 can be discarded, recovered and/or further processed for subsequent treatment and/or uses. In some embodiments, a skimming device can be used to harvest the lighter substances 256 floating on the surface of the liquid column while the heavier cellular mass and debris 258 can be harvested from the bottom of clarification tank 254. The remaining liquid 260 (e.g., water) can be filtered and returned to the growth chamber (recycled) or removed from the system (discarded).

In an embodiment in which the intracellular product 256 is oil (i.e., lipids), the oil can be processed into a wide range of products including vegetable oil, refined fuels (e.g., gasoline, diesel, jet fuel, heating oil), specialty chemicals, nutraceuticals, and pharmaceuticals, or biodiesel by the addition of alcohol. In some embodiments, intracellular products of interest can be harvested at any appropriate time, including, for example, daily (batch harvesting). In other embodiments, intracellular products are harvested continuously (e.g., a slow, constant harvest). The cellular mass and debris 258 can also be processed into a wide range of products, including biogas (e.g., methane, synthetic gas), liquid fuels (jet fuel, diesel), alcohols (e.g., ethanol, methanol), food, animal feed, and fertilizer.

In some embodiments, any suitable downstream treatment can be used in addition to gravity clarification. Possible downstream treatments are numerous and may be employed depending on the desired output/use of the intracellular contents and/or bio cellular mass and debris. For example, at 262 lipids 256 can be filtered by mechanical filters, centrifuges, or other separation devices, and then heated to evacuate more water. The lipids can then be further subjected to a hexane distillation at 264 or other refinement processes. In other embodiments, cellular mass and debris 258 can be subjected to gravity thickening at 266, anaerobic digestion at 268, a steam dryer or belt press at 270 for additional drying as necessary for food production, fertilizer production, etc. In some embodiments, anaerobic digestion of cellular mass and debris 258 can result in the recovery of biogases 272, CO₂ 274, and/or other nutrients 276 used during the growth phase or resulting from processing the slurry as discussed above.

Similar to various processes discussed above adapted to optimize the growth and/or flocculation of the algae slurry, in some embodiments, the separation of intracellular products 256 from cellular mass and debris 258 can be enhanced, controlled and/or optimized within clarification tank 254 by flocculating the cellular mass and debris. In such embodiments, cellular mass and debris 258 can be flocculated such that it agglomerates and settles more rapidly thus reducing the overall processing time for harvesting usable products from the algae slurry. In other embodiments, the efficiency with which the intracellular products 256 can be collected by, for example, skimming, can be enhanced by flocculating such intracellular products such that they agglomerate but continue to float on the surface. Flocculation of either intracellular products 256, cellular mass and debris 258 or both can be a chemical flocculation, an electro-flocculation or any other process that effectuates a similar agglomeration or coagulation of such products (e.g., intracellular products 256 and/or cellular mass and debris 258) as discussed above.

For example, in some embodiments, it is contemplated that the flocculation of either intracellular products 256, cellular mass and debris 258 or both can be monitored, controlled and/or optimized in accordance with zeta potential measurements by the addition of an emf or EMP as discussed previously. In such embodiments, it is contemplated that the process of flocculating such products is accomplished without chemical additives. In other words, a chemical free process is contemplated wherein the flocculation method comprises electro-flocculation. In other embodiments, additional methods common to those of skill in the art may be used to flocculate either intracellular products 256, cellular mass and debris 258 or both, including, but not limited to, chemical flocculation. In some embodiments, one flocculation technique may be employed while in other embodiments a combination of one or more electro- and/or chemical flocculation techniques can be employed together. In chemical based methods, zeta potential is still useful for determining the correct chemical coagulants and the proper dose of such coagulants and for monitoring flocculation as disclosed previously.

According to some embodiments, as mentioned above, in order to facilitate, control and/or optimize the flocculation and recovery of either intracellular products 256, cellular mass and debris 258 or both, zeta potential can be monitored and/or adjusted as necessary. In such embodiments, various samples of the slurry flow (whether before or after passing through extraction device 100) can be taken and tested using a zeta potential meter (not shown) common to those of skill in the art. Alternatively, one or more on-line streaming current device(s) (not shown but common to those of skill in the art), that have been calibrated using a zeta potential meter, can be located at any desirable location within the apparatuses depicted in FIG. 9 or used at any desirable time within the process described with reference to FIG. 9. In such embodiments, zeta potential can be measured, monitored and controlled in real time as the process described with reference to FIG. 9 progresses or transpires. Likewise, the state of charge, and changes within the state of charge, can be continuously monitored and quantified as the process transpires such that zeta potential can be adjusted as necessary or so that the optimum conditions for flocculation can be readily identified.

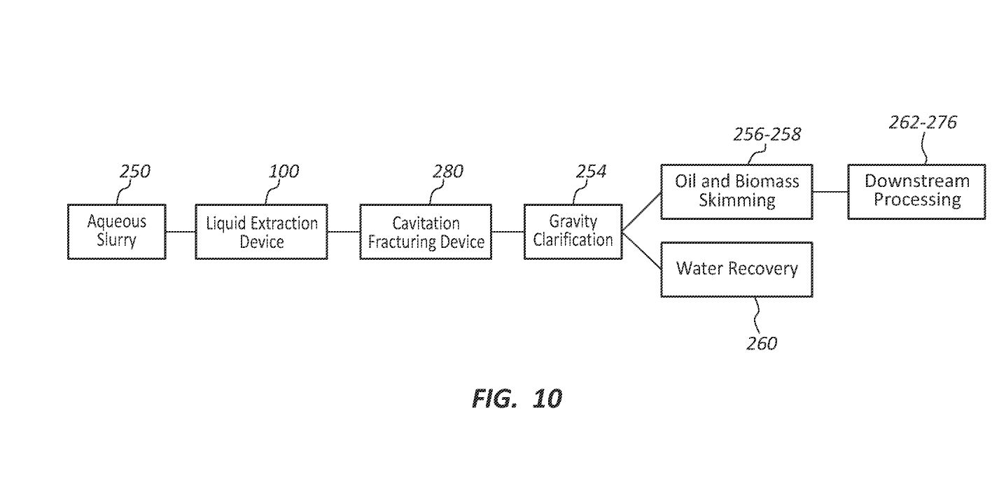

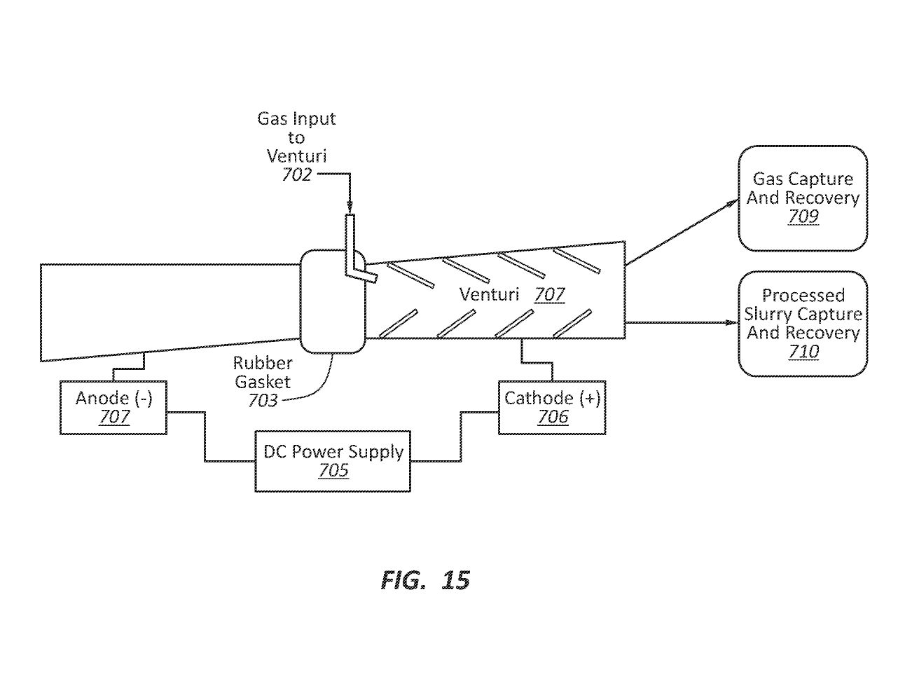

In some embodiments, a method of harvesting cellular mass and debris from an aqueous solution containing algae cells by subjecting algae cells to pulsed emf and to cavitation (i.e., microbubbles) in an apparatus as described herein, resulting in a mixture that includes both intracellular product(s) (e.g., lipids) and cellular mass and debris is contemplated. A process flow diagram that includes a cavitation step is shown in FIG. 10. The methods and apparatuses of this embodiment can use any of the lipid extraction devices described herein. In addition, the methods and apparatuses of such embodiments can be enhanced through the use of zeta potential as described elsewhere. In various embodiments, the algae cells can be subjected to cavitation before application of a (upstream of) pulsed emf or EMP, or they may be subjected to cavitation concomitantly with an EMP (see FIG. 15 that depicts the cavitation device electrified as it would be the EMP conductor).

With brief reference to FIG. 15, in some embodiments, a cavitation device includes an anode 704, a cathode 706, and venturi mixer 707 (all in one). In such embodiments, the cavitation unit is reduced (e.g., by half), a non-conductive gasket 703 is added, and it is electrified by power supply 705.

In some embodiments contemplating a cavitation step, a micron mixing device, such as a static mixer or other suitable device such as a high throughput stirrer, blade mixer or other mixing device is used to produce a foam layer composed of microbubbles within a liquid medium containing a previously lysed microorganism biomass. Any device suitable for generating microbubbles, however, can be used. Following micronization, the homogenized mixture begins to rise and float upwards. As this mixture passes upwards through the liquid column, the less dense valuable intracellular substances freely attach to the rising bubbles, or due to bubble collision, into a heavier sinking cellular mass and debris waste, (allowed to sink in some embodiments due to heated water specifics). The rising bubbles also shake loose trapped valuable substances (e.g., lipids) which also freely adhere to the rising bubble column. Once the foam layer containing these useful substances has risen to the top of the liquid column, they now can be easily skimmed from the surface of the liquid medium and deposited into a harvest tank for later product refinement. Once the foam layer rises to the top of the secondary tank, the water content trapped within the foam layer generally results in less than 10% (e.g., 5, 6, 7, 8, 9, 10, 10.5, 11%) of the original liquid mass. Trapped within the foam are the less dense useful substances, and the foam is easily floated or skimmed off the surface of the liquid medium. This process requires only dewatering of the foam, rather than evaporating the total liquid volume needed for conventional harvest purposes. This drastically reduces the dewatering process, energy or any chemical inputs while increasing harvest yield and efficiency as well as purity. In this method, water can be recycled to the growth chamber or removed from the system. Cellular mass and debris can be harvested at any appropriate time, including, for example, daily (batch harvesting). In other embodiments, cellular mass and debris is harvested continuously (e.g., a slow, constant harvest).

Once the liquid medium has achieved passage through the EMP apparatus, it is allowed to flow over into a secondary tank (or directly into a device that is located near the bottom of the tank). In this method of dewatering, the secondary tank is a tank containing a micron bubble device or having a micron bubble device attached for desired intracellular component separation and dewatering. After transmembrane lysis, a static mixer or other suitable device (e.g., any static mixer or device which accomplishes a similar effect producing micro-bubbles) is used and is located at the lowest point within a secondary tank. When activated, the static mixer produces a series of micron bubbles resulting in a foam layer to develop within the liquid medium. As the liquid medium is continuously pumped through the micro mixer, bubbled foam layers radiate outwards through the liquid and begin to rise and float upwards. The less dense desired intracellular components suspended within the liquid medium attach to the micron bubbles floating upwards and flocculate to the surface or are detached from heavier sinking biomass waste, (allowed to sink due to specific gravity alterations) due to rising bubble collision within the water column.

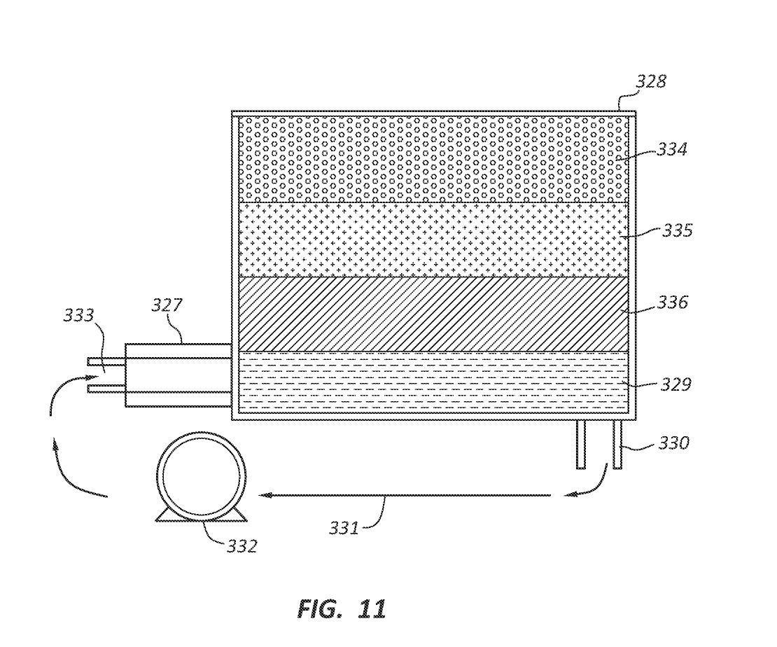

FIG. 11 illustrates a lower mounting location for a micron mixer 327 when in association with secondary tank 328 and containing a previously fractured biomass 329 suspended within a liquid medium according to some embodiments. This liquid medium is then allowed to flow through a lower secondary tank outlet 330 where it is directed to flow through conduit 331 having a directional flow relationship with a liquid pump 332. Due to pumping action, the liquid is allowed a single pass through, or to re-circulate through, the micron mixer via a micron mixer inlet opening 333. As liquid continues to flow through the micron mixer 327, microscopic bubbles 334 are produced which radiate outwards within the liquid column 335, forming a foam layer 336. As the process continues, the composed layer starts to rise upward toward the surface of the liquid column 335. Once the foam layer 336 starts its upward journey toward the surface of the liquid column 335, the pump 332 is shut down, and thus the micronization process is complete. This allows all micron bubbles 334 produced at the lower exit point of the micron mixer 327 to rise to the surface and, as they do, they start collecting valuable intracellular substances released into the liquid medium during the EMP process. This upward motion of the micron bubbles 334 also rubs or bumps into heavier downward-sinking cellular mass and debris, further allowing the release of trapped lighter valuable substances that have bonded with heavier-sinking cellular mass and debris. Once detached, these substances adhere to the micron bubbles 334 floating upwards towards the surface.

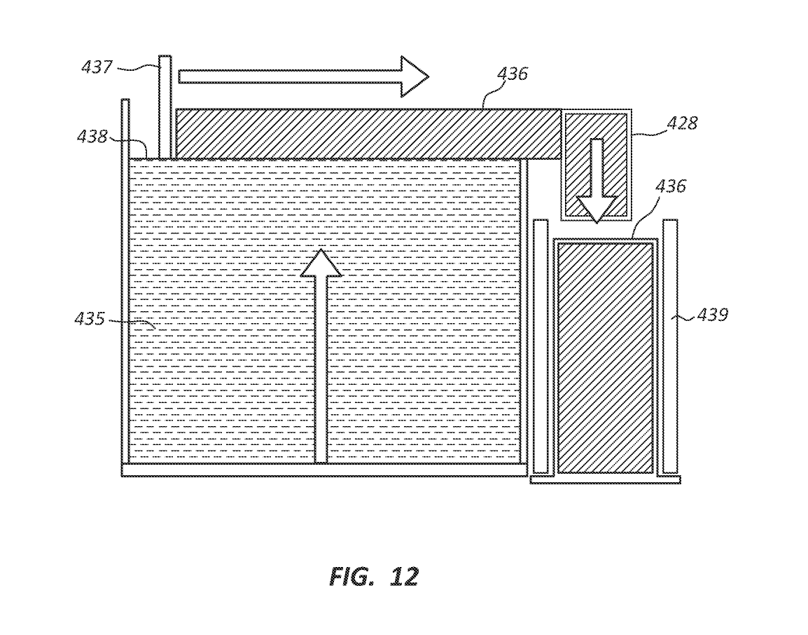

With reference now to FIG. 12, an illustration is provided to show a method for harvesting a foam layer 436 containing approximately ten percent of the original liquid medium mass/biomass 401. As the foam layer 436 containing the valuable intracellular internal substances rises to the surface of the liquid medium 435, a skimming device 437 can be used to remove the foam layer 436 from the surface 438 of liquid medium 435. The skimming device 437 located at the surface area of the secondary tank 428 allows the foam layer 436 to be pushed over the side wall of the secondary tank 428 and into a harvesting container 439 where the foam layer 436 is allowed to accumulate for further substance harvesting procedures.

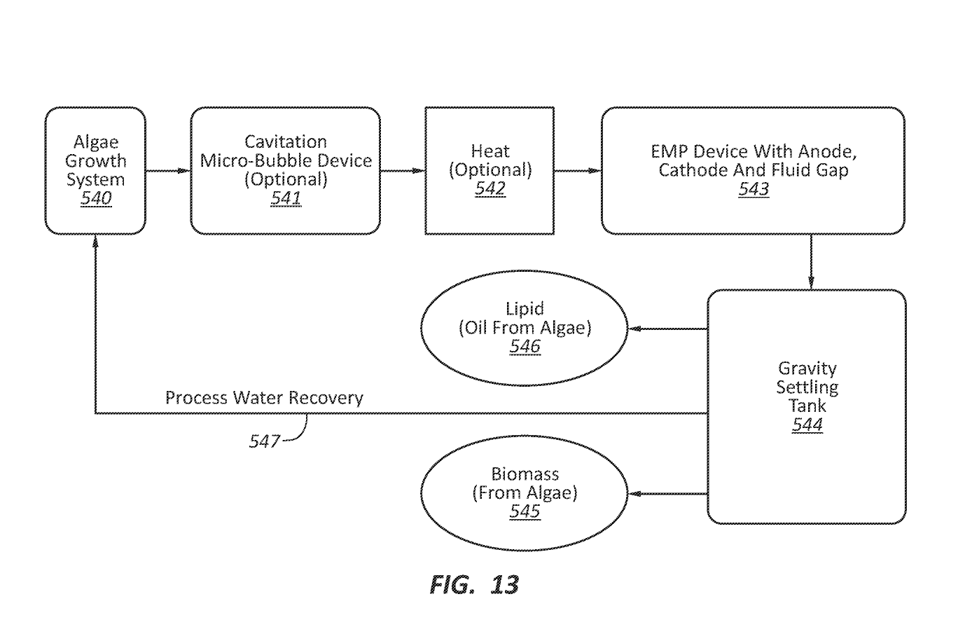

FIG. 13 illustrates one embodiment of a method and apparatus (system) as described herein for the harvest of useful substances from an algae biomass. Microorganism algae are grown in a containment system 540 and at the end of an appropriate growth cycle are transferred into the substance recovery process. The algae biomass are flowed through an optional micron bubble cavitation step 541, used to soften the outer cellular wall structure prior to other bio substance recovery processes.

With continued reference to FIG. 13, in some embodiments, an optional heat process 542 can be applied to change the gravity specifics of the liquid feed stock water containing the biomass. The heat option 542 allows a faster transfer of particular substances released during the harvest process. After the biomass has reached an appropriate heat range, it is then allowed to flow through an electromagnetic pulse field, the EMP station 543 where transiting biomass cells are exposed to the electromagnetic transfers resulting in the fracturing of the outer cellular wall structures.

According to some embodiments, once the fractured biomass is flowed through the EMP process 543, it transitions into a gravity clarifier tank 544 where heavier matter (ruptured cell debris/mass) 545 sinks down through the water column as the lighter matter (intracellular products) 546 rises to the surface where it facilitates an easier harvest. The heavier sinking mass 545 gathers at the bottom of the clarifier tank 544 where it can be easily harvested for other useful substances. After substance separation and recovery, the remainder of the water column 547 is sent through a water reclaiming process and, after processing, is returned back into the growth containment system 540.

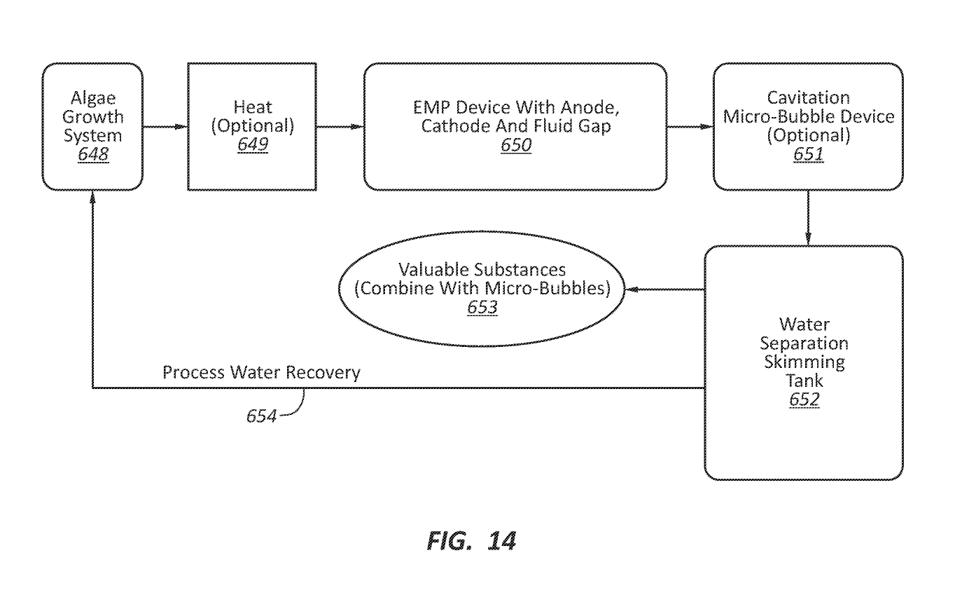

FIG. 14 illustrates another embodiment of a method and apparatus (system) as described herein for the harvest of useful substances from an algae biomass. Microorganism algae are grown in a containment system 648 and at the end of an appropriate growth cycle are then transferred into the substance recovery process. The substance recovery consists of the algae biomass being transferred into an optional heat process 649 where the biomass water column is optionally subjected to heat prior to the EMP station 650. After the EMP process, the fractured biomass is then optionally transferred over into a cavitation station 651 where micron bubbles are introduced at a low point in a water column containment tank 652. As the microbubbles rise through the water column, the valuable released bio substances (intracellular products) 653 attach to the rising bubbles which float to the surface of the water column allowing an easier and faster skimming process for substance recovery. After substance recovery, the remainder of the water column is sent through a water reclaiming process 654 and, after processing, is returned back into the growth system 648.

As mentioned above, the various methods and apparatuses of the embodiments disclosed and discussed above, including those associated with FIG. 1 through 15, can use any of the component apparatuses and/or associated methods described herein. In some embodiments, each of the apparatuses and/or methods disclosed herein can be used together in a single system. In other embodiments, as few as one of the apparatuses and/or methods disclosed herein can be used is isolation to accomplish one or more discrete functions. Moreover, the methods and apparatuses of the various embodiments disclosed herein can be enhanced and/or optimized through the use of zeta potential as described in various locations throughout this application.

### Supervisory Control and Data Acquisition

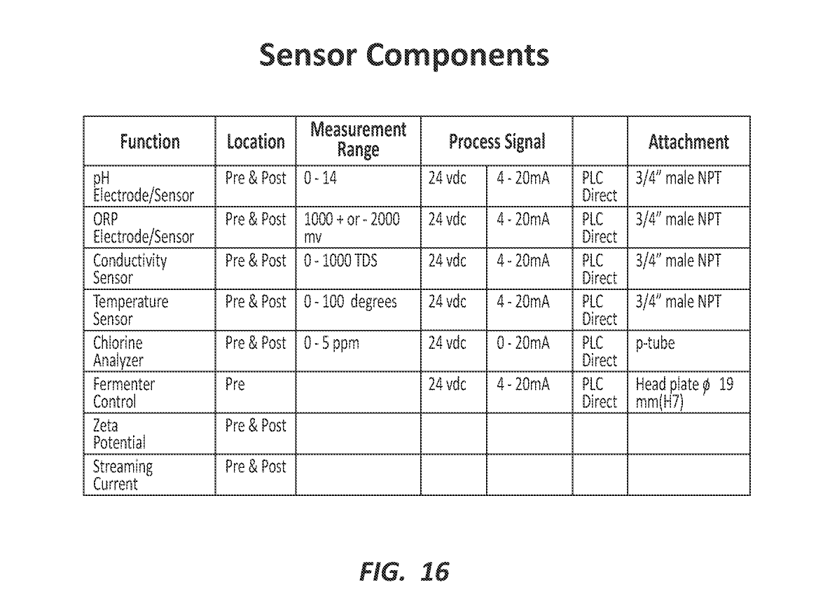

In some embodiments, the forgoing methods, systems and apparatuses involve the development and deployment of a specially selected array of sensor probes, which communicate among/between each other via Supervisory Control and Data Acquisition (SCADA) technology, and to a control module, power supplies and power conditioning units, such as pulse and frequency generators/modulators for the anode and cathode pair or zeta potential meter(s) or streaming current device(s), etc. Various sensor probes and/or related devices according to some embodiments are identified in FIG. 16 and will be discussed in greater detail below.

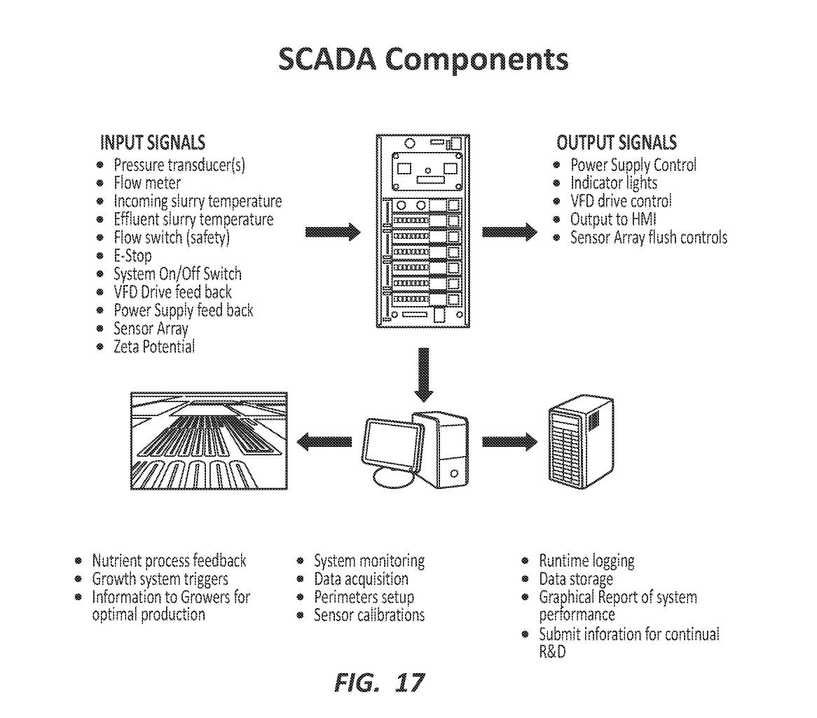

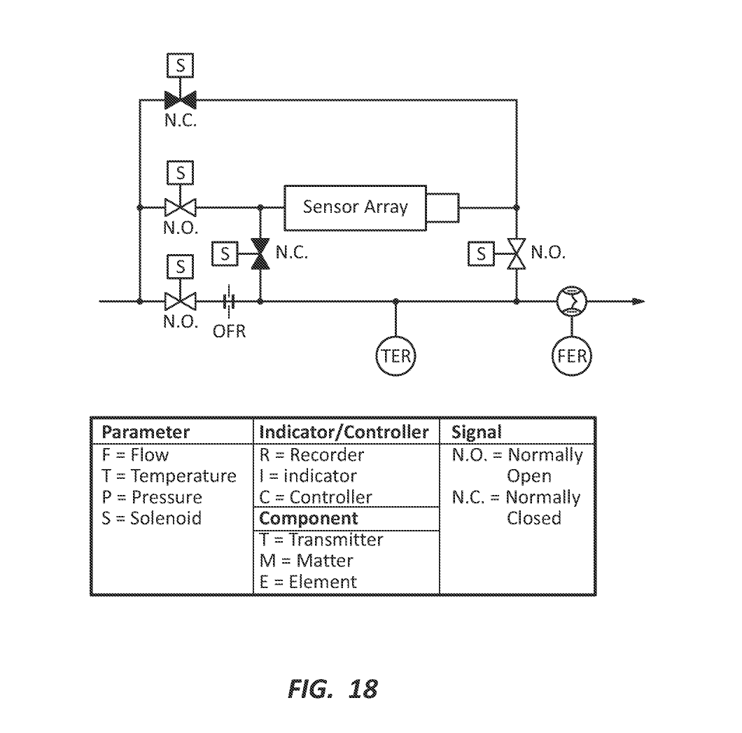

The sensor measurement parameters, according to some embodiments, are shown in FIG. 17. Some embodiments measure parameters comprising, among other things, water hardness, pH, ORP, conductivity, zeta potential, streaming current, and/or streaming potential where the dielectric properties may be quantified, and may be compared to cell density. In some embodiments, dissolved gas such as chlorine, ammonia, hydrogen, oxygen, CO₂, and other process and/or waste gas values/volumes can be used for process control and monitoring or enhancing algae growth. In yet other embodiments, additional parameters, such as temperature, are measured such that corresponding data can be used for process control. In some embodiments, process control comprises growth of algae, processing, separation and/or extraction, as well as handling the effluent, recirculation and return to process. FIG. 18 illustrates a non-limiting example of a system according to some embodiments of the invention.

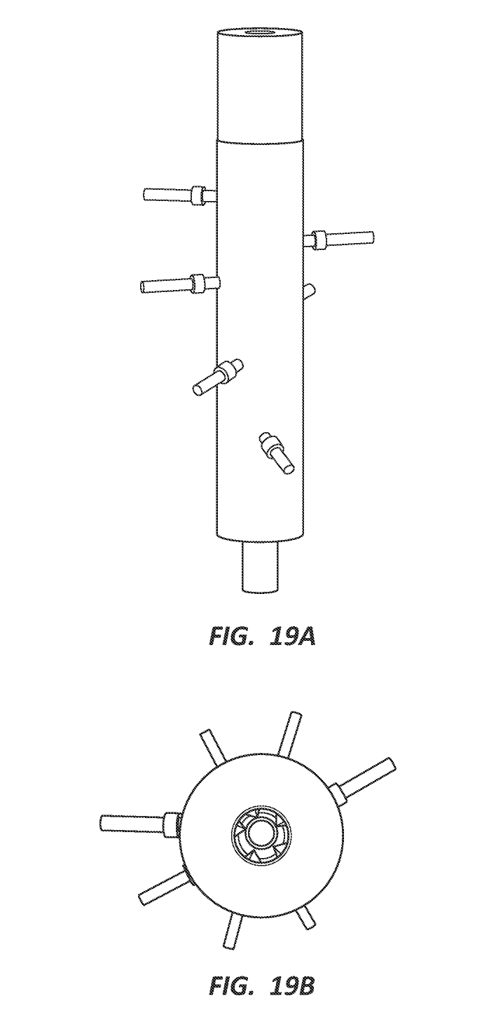

An example of a sensor array according to some embodiments is shown in FIGS. 19A and 19B. Some embodiments of a sensor array comprise a spool piece. In some embodiments, the spool piece comprises a flow inlet. In some embodiments, the flow inlet comprises a spiraling foot. According to some embodiments, the spiraling foot may be structured to initiate a clockwise or counterclockwise flow. According to some embodiments, the clockwise or counterclockwise flow may allow the working/process fluid to move past at least one instrument probe to provide a fresh sample presentation to the instrument probe(s). In some embodiments, a series of instrument probes (e.g., at least two probes) may be staged in sequence. In some embodiments, a series of instrument probes (e.g., at least two probes) may be staged in a helix design. In some embodiments, a series of instrument probes (e.g., at least two probes) may be spaced relative to each other to resists the creation of turbulent flow within the spool.

Some embodiments may comprise a flow straightener, straightening vein, berms and/or undulations. Some embodiments may comprise a plurality of flow straighteners, straightening veins, berms or undulations. In some embodiments, at least one of the flow straightener, straightening veins, berms and/or undulations may be structured to direct flow in these directions as well.

Some embodiments comprise at least one outlet section in fluid connection to a spool piece. Some embodiments may comprise a plurality of outlet sections in fluid connection to a spool piece. Some embodiments of the outlet section comprise at least one flow restriction appliance or device. In some embodiments, the at least one flow restriction appliance may be structured to ensure the chamber of the spool can be filled with working/process fluid at all times in all positions, while flow has been established.

Some embodiments comprise elements structured to provide back flushing or chemical cleaning as shown in FIGS. 19A and 19B.

Some embodiments comprise central control of the dynamic flow condition inside a spool piece. Other embodiments comprise local control of the dynamic flow condition inside a spool piece. Some embodiments are structure to be used as “indication only” of the dynamic flow condition inside a spool piece. The system can also be filled with working fluid as a static/grab sampling and analytical tool, for point measurements. An example of this would be to characterize the composition of feed water in open or closed photo bioreactors, ponds or raceways in various stages of growth, maintenance, and operation. The system can also be deployed and connected to remote telemetry or local indications of water chemistry and algae culture. Lysimiter, and separate affects testing can also be accomplished remotely and unmanned, with the capability of both static and dynamic change in state scenarios. Some embodiments comprise battery operation of sensors, facilitating both local and central control. All of these configuration support data acquisition, and central signal distribution from the sending units, where instructions for set points can be modified and executed for range, and functionality, such as preset and resetting local and central alarm control, and calibration.

As mentioned above, FIG. 16 illustrates non-limiting examples of the types of sensors that may be used according to some embodiments. Sensors may be used to detect pH, ORP, TDS, temperature, conductivity, salinity, chlorine, dissolved oxygen, cell density, CO₂, zeta potential, streaming current, streaming potential and/or ammonia. An individual sensor may be used, or multiple sensors may be used. Direct probe information may be used to detect pH, ORP, TDS, temperature, conductivity, salinity, chlorine, dissolved oxygen, cell density, CO₂, zeta potential, streaming current, streaming potential and/or ammonia. In some embodiments, multiple inputs may be used to detect pH, ORP, TDS, temperature, conductivity, salinity, chlorine, dissolved oxygen, cell density, CO₂, zeta potential, streaming current, streaming potential and/or ammonia.

In some embodiments, a probe or multiple probes may be mounted via a wet-tap system to withstand a minimum of 50 psi. In some embodiments, mounting of at least one probe can be done with threaded taps and a threaded body probe and/or a compression nut system over a smooth body probe.

In some embodiments, the chemical composition of algae or other substrate may be analyzed up and/or downstream to match electrode compatibility. A higher salt content may require different electrodes and different housing than a fresh water solution.

In some embodiments, zeta potential, streaming potential and/or streaming current may be analyzed up and/or downstream to optimize processing of the algae at various stages.

Some embodiments comprise a physical housing and/or sensor array structure. Some embodiments of a physical housing and/or array structure may comprise: a housing to maintain high flow while minimizing probe fouling; interior surfaces to be of a non-fouling material by means of a polished metal, ceramics or a coating that will deter formation of bio-residues; a housing to remediate air trapped and evacuation from the system; a system for bypassing the main flow for cleaning and calibrating probes; probes mounted on a helix to decrease and/or eliminate eddy currents in order to maintain a virgin sampling medium; and an array to be mounted upstream and downstream within the system.

Some embodiments comprise measurements, triggers and/or algorithmic maps for SCADA applications. Some embodiments comprise sensors connected to a SCADA system. Some embodiments comprise sensors connected to a SCADA system structured to modify the system's voltage, amperage, pulse frequency, amplitude and/or flow rates for optimal growth and/or flocculation based on a predefined series of process maps. Some embodiments comprise algorithms designed to measure point-to-point changes between upstream and post system process. Some embodiments comprise at least one probe utilized for taking at least one point measurement. Some embodiments comprise several probes structured to take at least one point measurement. Some embodiments comprise probes structured to take point-to-point comparisons (e.g., ORP).

Some embodiments comprise real time reading of probes with sampling taken on point of change. Some embodiments comprise exporting information on time intervals. Some embodiments comprise exporting information with a “dead-band” of a +/−percentage to ignore extra data points in the case of small fluctuations from system noise, wiring, air bubbles in system, etc. Some embodiments comprise probe averaging for fast response probes on 50 or 100 ms intervals. For example, some embodiments comprise probes which read conductivity every 5 ms. As a non-limiting example, if an average of 100 ms is used, a total of 20 sample points may be averaged into a single data point. This reduces database size and false readings. Some embodiments comprise separate algorithms, which run parallel to the system process, designed to predict probe changes and monitor for irregular probe behavior. Some embodiments comprise a fault flag set via software/hardware to alert an operator to evaluate, correct and clear the fault. Some embodiments are structured to stream all information to a database server for evaluation and graphical presentation.

Some embodiments comprise systems structured for cleaning and/or antifouling. Some embodiments comprise a point injection system for cleaning of probes (e.g., argon or CO₂ blasts on point spots). Some embodiments comprise a manifold structured with a bypass valve for servicing and/or cleaning. Some embodiments comprise, in conjunction with a bypass loop, a system of valves structured, when actuated, to reverse the process flow through the spool backwashing areas of biomass/particulate build up. In some embodiments, all probes/and or some of the probes will still function properly during the backwash phase. In some embodiments, downstream probes will tend to foul faster than upstream ones, therefore special care may be utilized for cleaning. In some embodiments, alternatives to water/chemical/gas cleaning may be utilized. For example ultrasonic emissions during a high pressure rinse may be utilized with some embodiments. Further, some embodiments may utilize a chemical that would emulsify, via ultrasonics, oils and contaminants to assist in the cleaning of probes.

As mentioned above, FIG. 17 illustrates SCADA components which may be utilized individually or in combination with each other according to some embodiments. Some embodiments comprise growth systems which may be structured to utilize nutrient process feedback, growth system triggers and information to growers for optimal production. For example, zeta potential can be utilized in with some embodiments. Some embodiments comprise an HMI display(s) comprising: system monitoring, data acquisition, perimeters setup and/or sensor calibrations. Some embodiments comprise database(s) comprising runtime logging, data storage, graphical report of system performance and/or additional information to be used for research and development.

FIG. 18 illustrates a non-limiting example of a system according to some embodiments. FIG. 18 is shown with the valves in normal operation. By changing the valve positions, flow may be redirected to the outlet side of the spool, thus flowing backward causing particle build up from the “normal” direction to be flushed out while still passing fluid over the sensor array. Flushing periods may be based on speed of medium passed through the spool and types of particulates in solution.

Some embodiments comprise at least one OFR (Orifice Flow Restrictor) which may be structured to divert 15 to 30 percent of the medium to the spool for sampling. Some embodiments comprise at least one flow meter attached to the end of the system that is used in conjunction with the flocculation system to control pump output and log process volumes. Some embodiments comprise at least one sensor array which is a proprietary spool type sensor array installed either upstream, downstream or on both sides of the flocculation equipment for monitoring and calibration of the power and flow delivery. In some embodiments, installation may be in a no-turbulent zone of the process. In some embodiments, locations may be a minimum of 12″ away from any bend or piping restriction and at least 24″ to 36″ from any pump. In some embodiments sensors may be mounted vertically or horizontal in such a fashion to not allow air to get trapped in the system. Some embodiments are structured to have a 20 GPM max flow rate. Some embodiments are structured to not exceed 50 PSI.

As illustrated in FIG. 18, some embodiments comprise wiring specifications comprising: a 4 to 8 sensor array; a 4-20 ma output signal-self powered (powered from sensor); cabling from a sensor junction into a common NEMA 4×PVC enclosure with a single12pin connection (Amphenol #PTOOE-14-12P or equal) with an individual shield 18 awg-6 pr trunk cable back to the SCADA system; a backwash system incorporated across the spool utilizing the process fluid in a reverse direction at predefined time intervals based on fluid speed (e.g., a slower flow rate allows deposits to accumulate faster, thus a more frequent flushing interval is required); and a valve sequencing controlled by a PLC system.

Thus, as discussed herein, various embodiments of the present invention embrace systems, apparatuses and methods for harvesting cellular mass and debris as well as intracellular products from algae cells which can be used as a substitute for fossil oil derivatives in various types of product manufacturing. The present invention further embraces systems, apparatuses and methods for identifying and measuring key parameters of water and gas chemistry in which algae cells can grow and be mass produced such that when the algae cells mature they are separable from the water and the algae cells can be fractured in order to separate cellular mass and debris from various intracellular products using pulsed electromotive forces or electromagnetic pulses and other methods, including mechanical and/or chemical.

The SCADA system previously described is, according to some embodiments, adapted to facilitate identifying, measuring and controlling key parameters in relation to other biomass developing processes and bio-refining processes so as to maximize the efficiency and efficacy of such processes while standardizing the underlying parameters to facilitate and enhance large-scale production of bio-based products and/or bio-energy.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A method for processing standardized biomass feedstocks to yield bio-based products, comprising:
providing a standardized biomass feedstock;
formatting the standardized biomass feedstock for subsequent refinement; and
processing the standardized biomass feedstock to yield bio-based products therefrom.

2. The method of claim 1, wherein providing a standardized biomass feedstock comprises providing one of: a terrestrial biomass feedstock and a high moisture content biomass feedstock.

3. The method of claim 2, wherein the terrestrial biomass feedstock comprises one of: a herbaceous biomass feedstock, a woody biomass feedstock, an agricultural food, an agricultural feed crop, an agricultural crop waste, an agricultural residue, a wood waste, a wood residue, an aquatic plant, a vegetable oil, a livestock manure, a municipal waste, and an industrial waste.

4. The method of claim 2, wherein the high moisture content biomass feedstock comprises one of: algae, beet pulp, and sludge.

5. The method of claim 1, wherein providing a standardized biomass feedstock comprises providing a standardized mixture of constituent biomass feedstock components.

6. The method of claim 5, wherein the mixture of constituent biomass feedstock components comprises a base feedstock combined with one of: an algae feedstock, an herbaceous feedstock, a woody feedstock, an additive, an add mixture, a bacteria, a catalyst, a binder, a chemical coagulant, a chemical flocculant, moisture, inorganic components, and water.

7. The method of claim 1, wherein formatting the standardized biomass feedstock for subsequent refinement comprises combining constituent biomass feedstocks.

8. The method of claim 7, wherein the constituent biomass feedstocks comprise a base feedstock combined with one of: an algae feedstock, an herbaceous feedstock, a woody feedstock, an additive, an add mixture, a bacteria, a catalyst, a binder, a chemical coagulant, a chemical flocculant, moisture, inorganic components, and water.

9. The method of claim 1, wherein formatting the standardized biomass feedstock for subsequent refinement comprises processing the biomass feedstock such that it assumes a specified morphology selected from the group comprising: a liquid, a gas, a powder, a dust, a residue, a concentrate, a briquette, a pellet, a tablet, and a combustible.

10. The method of claim 1, wherein processing the standardized biomass feedstock to yield bio-based products therefrom comprises one of: anaerobic digestion, dry fractionation, solvent fractionation, fermentation, gasification, transesterification, hydrothermal upgrading, pyrolysis, torrefaction, catalytic hyrdotreating, Fischer-Tropsch synthesis, hydroforming, enzyme hydrolysis, hydrocracking, and co-firing.

11. A method for standardizing biomass feedstocks for use in large-scale bio-refinement, comprising:
providing a base biomass feedstock; and
mixing the base biomass feedstock with a secondary component to provide a standardized biomass feedstock mixture for us in large-scale bio-refinement.

12. The method of claim 11, wherein the base biomass feedstock comprises one of: a terrestrial biomass feedstock and a high moisture content biomass feedstock.

13. The method of claim 12, wherein the secondary component comprises one of: an algae feedstock, an herbaceous feedstock, a woody feedstock, an additive, an add mixture, a bacteria, a catalyst, a binder, a chemical coagulant, a chemical flocculant, moisture, inorganic components, and water.

14. The method of claim 13, wherein the base biomass feedstock and the secondary component are mixed at a ratio that is selected from a group comprising: about 1:1, about 1:10, about 1:100, about 1:1000, about 10:1, about 10:100, about 10:1000, about 100:1, about 100:10, about 100:1000, about 1000:1, about 1000:10, and about 1000:100.

15. The method of claim 13, further comprising combining the base biomass feedstock and the secondary component with a tertiary component, wherein the tertiary component comprises one of: an algae feedstock, an herbaceous feedstock, a woody feedstock, an additive, an add mixture, a bacteria, a catalyst, a binder, a chemical coagulant, a chemical flocculant, moisture, inorganic components, and water.

16. The method of claim 15, wherein the base biomass feedstock, the secondary component, and the tertiary component are mixed at a ratio that is selected from a group comprising: about 1:1:1, about 1:1:10, about 1:1:100, about 1:1:1000, about 1:10:1, about 1:10:10, about 1:10:100, about 1:10:1000, about 1:100:1, about 1:100:10, about 1:100:100, about 1:100:1000, about 1:1000:1, about 1:1000:10, about 1:1000:100, about 1:1000:1000, about 10:1:1, about 10:1:10, about 10:1:100, about 10:1:1000, about 10:10:1, about 10:10:100, about 10:10:1000, about 10:100:1, about 10:100:10, about 10:100:100, about 10:100:1000, about 10:1000:1, about 10:1000:10, about 10:1000:100, about 10:1000:1000, about 100:1:1, about 100:1:10, about 100:1:100, about 100:1:1000, about 100:10:1, about 100:10:10, about 100:10:100, about 100:10:1000, about 100:100:1, about 100:100:10, about 100:100:1000, about 100:1000:1, about 100:1000:10, about 100:1000:100, about 100:1000:1000, about 1000:1:1, about 1000:1:10, about 1000:1:100, about 1000:1:1000, about 1000:10:1, about 1000:10:10, about 1000:10:100, about 1000:10:1000, about 1000:100:1, about 1000:100:10, about 1000:100:100, about 1000:100:1000, 1000:1000:1, 1000:1000:10, and 1000:1000:100.

17. A method for extracting and recovering lipids from a biomass feedstock, comprising:
providing a biomass feedstock in a flowing aqueous slurry;
extracting at least a portion of intracellular lipids from the biomass feedstock; and
recovering at least a portion of the intracellular lipids extracted from the biomass feedstock.

18. The method of claim 17, further comprising the step of flocculating the biomass feedstock prior to extracting the intracellular lipids therefrom.

19. The method of claim 18, wherein the step of flocculating the biomass feedstock comprises applying a first electromotive force to the aqueous slurry.

20. The method of claim 19, wherein extracting at least a portion of the intracellular lipids is carried out by applying a second electromotive force to the biomass feedstock in the aqueous slurry.
